(19) United States
(12) Patent Application Publication
(10) Pub. No.: US 2025/0188152 A1
(43) Pub. Date: Jun. 12, 2025

(54) TRANSIENT EXPRESSION OF THERAPEUTIC PROTEINS

(71) Applicant: Bristol-Myers Squibb Company, Princeton, NJ (US)

(72) Inventors: Kitty AGARWAL, Princeton, NJ (US); Juan Camilo GONZALEZ RIVERA, Princeton, NJ (US); Todd William RYDER, Princeton, NJ (US); Anurag KHETAN, Princeton, NJ (US)

(73) Assignee: Bristol-Myers Squibb Company, Princeton, NJ (US)

(21) Appl. No.: 18/844,814

(22) PCT Filed: Mar. 9, 2023
(86) PCT No.: PCT/US2023/064035

§ 371 Date: Sep. 6, 2024

Related U.S. Application Data
(60) Provisional application No. 63/322,472, filed on Mar. 22, 2022.
(60) Provisional application No. 63/318,370, filed on Mar. 9, 2022.

(51) Int. Cl. C07K 16/00; C12M 1/00; C12M 1/42; C12M 3/06; C12N 15/85

(52) U.S. Cl. CPC C07K 16/00; C12M 27/16; C12M 29/10; C12M 35/02; C12M 47/10; C12N 15/85; C12N 2800/107

(57) ABSTRACT

The present disclosure provide novel methods of large-scale production of recombinant proteins, e.g., therapeutic proteins such as antibodies, comprising concentrating an eukaryotic cell culture to a high density and transiently transfecting the eukaryotic cells with a polynucleotide encoding the recombinant protein using electroporation, e.g., flow electroporation. In some aspects, the culture is performed under perfusion conditions using, e.g., a tangential flow filtration method such as alternating tangential flow filtration. The proteins obtained using the disclosed methods are comparable to those produced using stable transfection. The methods disclosed herein can be used, for example, to accelerate therapeutic agent development, to reduce host cell toxicity, or for individualized therapeutics such as small scale manufacturing of treatments for rare or orphan diseases.

### CROSS REFERENCE TO RELATED APPLICATIONS

This PCT application claims the priority benefit of U.S. Provisional Application Nos. 63/318,370, filed Mar. 9, 2022, and 63/322,472, filed Mar. 22, 2022, each of which is incorporated herein by reference in its entirety.

### FIELD

The present application relates to the field of transient gene expression of therapeutic recombinant proteins.

### BACKGROUND

With the increasing need to shorten timelines and improve throughput in recombinant protein production, there is a critical need to generate drug substance material rapidly and with product quality profile comparable to material produced in traditional, stable cell lines. Transient gene expression (TGE) potentially fills this gap by rapidly and consistently producing proteins of interest that are representative of clonal material. The ability of TGE to generate material weeks earlier than stable cell line production representative of clonal material, accelerates the timeline to clinical manufacturing by supporting downstream, formulation, and analytical development. Kelley (2020) Nat. Biotechnol. 38 (5): 540-545; Coffman et al. (2008) Biotechnol. Bioeng. 100 (4): 605-618; Bolisetty et al. (2020) MAbs 12 (1): p 176372. Thus, development programs can be carried out with minimal risk on downstream activities before final cell lines are chosen. Additionally, TGE enables earlier toxicological studies with material from the same mammalian host system as in later development stages, making assessments between program phases easier and more accurate. Further uses of TGE include small-scale manufacturing for rare or orphan diseases, e.g., for generation of small drug batches allowing the design of targeted medicines for specific groups of patients or even for individualized therapeutics (Gutierrez-Granados et al. (2018) Crit. Rev. Biotechnol. 38 (6): 918-940), enables more efficient production of orphan diseases therapeutics because it supports production versatility with reduced development needs, which decreases the global cost of treatment per patient (Sun et al. (2017) Am. J. Med. Genet. A 173 (9): 2307-2322). However, low productivity (low titers), product quality differences to clonal material, and scalability have limited the use of TGE to non-GLP preclinical studies and prevented the wide-spread application of transient expression in clinical development.

### BRIEF SUMMARY

The present disclosure provides a method for high-yield and/or high quality production of a recombinant protein in a large scale bioreactor comprising
(i) culturing eukaryotic cells to concentrate the eukaryotic cells to a high density; and
(ii) transiently transfecting the eukaryotic cells with a polynucleotide encoding the recombinant protein using electroporation.

In some aspects, the quality of the recombinant protein is comparable to that of clonal material, i.e., recombinant protein obtained using a stably transfected cell. In some aspect, culturing eukaryotic cells to concentrate the eukaryotic cells to a high density comprises maintaining the cells in an optimal growth phase.

In some aspects, the eukaryotic cells are mammalian cells. In some aspects, the mammalian cells are selected from the group consisting of CHO, VERO, BHK, HEK, HeLa, COS, MDCK, and hybridoma cells. In some aspects, the mammalian cells are CHO cells. In some aspects, the recombinant protein comprises an antibody or an antigen-binding portion thereof. In some aspects, the antibody or antigen-binding fragment binds an antigen selected from the group consisting of PD-1, PD-L1, CVTLA-4, LAG-3, TIGIT, GITR, CXCR4, CD73, HER2, VEGF, CD20, CD40, CD11a, tissue factor (TF), PSCA, IL-8, EGFR, HER3, and HER4. In some aspects, the bioreactor is a high-throughput bioreactor. In some aspects, the high-throughput bioreactor is an AMBR®250 or an AMBR®15 bioreactor. In some aspects, the bioreactor is a 1 L, 2 L, 5 L, 10 L, 25 L, 50 L, 100 L, 500 L, 1000 L, 2000 L, 5000 L, 10,000 L, or 20,000 L bioreactor. In some aspects, the bioreactor is a fed-batch production bioreactor. In some aspects, the bioreactor is a single use bioreactor. In some aspects, the bioreactor is a glass tank bioreactor.

In some aspects, the culturing, e.g., prior to transfection, is perfusion culture. In some aspects, the perfusion is conducted by tangential flow filtration. In some aspects, the tangential flow filtration is alternating tangential flow filtration (ATF). In some aspects, the perfusion is conducted using a REPLIGEN™ ATF2 system. In some aspects, the cells are concentrated to a density of at least about 40×10⁶, at least about 50×10⁶, at least about 60×10⁶, at least about 70×10⁶, at least about 80×10⁶, at least about 90×10⁶, at least about 100×10⁶, at least about 110×10⁶, at least about 120×10⁶, at least about 130×10⁶, at least about 140×10⁶, at least about 150×10⁶, at least about 160×10⁶, at least about 170×10⁶, at least about 180×10⁶, at least about 190×10⁶, or at least about 200×10⁶ cells/mL. In some aspects, the cells are concentrated to a density of about 40×10⁶ to about 200×10⁶, about 60×10⁶ to about 200×10⁶, about 80×10⁶ to about 200×10⁶, about 100×10⁶ to about 200×10⁶, about 120×10⁶ to about 200×10⁶, about 140×10⁶ to about 200×10⁶, about 160×10⁶ to about 200×10⁶, about 180×10⁶ to about 200×10⁶, about 40×10⁶ to about 180×10⁶, about 60×10⁶ to about 180×10⁶, about 80×10⁶ to about 180×10⁶, about 100×10⁶ to about 180×10⁶, about 120×10⁶ to about 180×10⁶, about 140×10⁶ to about 180×10⁶, about 160×10⁶ to about 180×10⁶, about 40×10⁶ to about 160×10⁶, about 60×10⁶ to about 160×10⁶, about 80×10⁶ to about 160×10⁶, about 100×10⁶ to about 160×10⁶, about 120×10⁶ to about 160×10⁶, about 140×10⁶ to about 160×10⁶, about 40×10⁶ to about 140×10⁶, about 60×10⁶ to about 140×10⁶, about 80×10⁶ to about 140×10⁶, about 100×10⁶ to about 140×10⁶, about 120×10⁶ to about 140×10⁶, about 40×10⁶ to about 120×10⁶, about 60×10⁶ to about 120×10⁶, about 80×10⁶ to about 120×10⁶, about 100×10⁶ to about 120×10⁶, about 40×10⁶ to about 100×10⁶, about 60×10⁶ to about 100×10⁶, about 80×10⁶ to about 100×10⁶ cells/mL. In some aspects, the cells are concentrated to a density of about 100×10⁶, about 110×10⁶, about 120×10⁶, about 130×10⁶, about 140×10⁶, about 150×10⁶, about 160×10⁶, about 170×10⁶, about 180×10⁶, about 190×10⁶, about 200×10⁶, about 210×10⁶, about 220×10⁶, about 230×10⁶, about 240×10⁶, or about 250×10⁶ cells/mL.

In some aspects, electroporation is conducted at a cell density of about 100×10⁶, about 110×10⁶, about 120×10⁶, about 130×10⁶, about 140×10⁶, about 150×10⁶, about 160×10⁶, about 170×10⁶, about 180×10⁶, about 190×10⁶, about 200×10⁶, about 210×10⁶, about 220×10⁶, about 230×10⁶, about 240×10⁶ or about 250×10⁶ cells/mL. In some aspects, the electroporation is flow-based electroporation. In some aspects, electroporation is conducted using a MAXCYTE™ transfection system. In some aspects, the transfection system is a MAXCYTE™ STX or VLX transfection system. In some aspects, electroporation is conducted at a DNA to cell ratio of about 1, about 1.1, about 1.2, about 1.3, about 1.4, about 1.5, about 1.6, about 1.7, about 1.8, about 1.9, about 2, about 2.1, about 2.2, about 2.3, about 2.4, or about 2.5 μg DNA per 1×10⁶ cells.

In some aspects, the post-transfection cell culture start density is between 5×10⁶ and 20×10⁶ cells/mL. In some aspects, the post-transfection cell culture is shifted temperature for some of the culture period. In some aspects, the temperature of the post-transfection cell culture is shifted for some of the culture period. In some aspects, the post-transfection cell culture comprises a temperature shift on Day 1 or Day 2. In some aspects, the temperature shift is from about 37° C. to about 32° C. In some aspects, the temperature shift is from about 37° C. to about 34° C. In some aspects, the post-transfection cell culture comprises the addition of N,N-dimethyl acetamide (DMA). In some aspects, DMA is added at about 0.125% v/v or about at 0.250% v/v. In some aspects, DMA is added at between about 0.125% v/v to about 0.250% v/v. In some aspects, the post-transfection cell culture comprises the addition of sodium butyrate (NaBu.). In some aspects, NaBu is added at a concentration between about 1 mM and about 2 mM.

In some aspects, protein yield of the method is at least about 0.1 g/L, at least about 0.2 g/L, at least about 0.3 g/L, at least about 0.4 g/L, at least about 0.5 g/L, at least about 0.6 g/L, at least about 0.7 g/L, at least about 0.8 g/L, at least about 0.9 g/L, at least about 1 g/L, at least about 2 g/L, at least about 3 g/L, at least about 4 g/L, at least about 5 g/L, or at least about 6 g/L after 7 days of culture. In some aspects, the protein yield is up to 2 g/L after 14 days of culture.

The present disclosure a method for high-yield and/or high quality production of a recombinant protein in a bioreactor comprising
(i) culturing eukaryotic cells under continuous ATF perfusion prior to transfection,
(ii) concentrating the cells to a density of 40-200×10⁶ cells/mL using continuous ATF perfusion prior to transfection;
(iii) transiently transfecting the eukaryotic cells with a polynucleotide encoding the recombinant protein using flow-based electroporation; and.
(iv) post-transfection culturing the transiently transfected eukaryotic cells.

In some aspects, the method produces recombinant protein with product quality comparable to clonal material. In some aspects, post-transfection culturing comprises (i) a cell culture start density of about 15×10⁶ cells/mL, (ii) DMA at about 0.125% (v/v/), (iii) NaBu at about 1 mM, and (iv) a temperature shift at Day 1 from about 36.5° C. to about 32° C., wherein the volume of the bioreactor is between about 250 mL and about 5 L. In some aspects, the values of product quality attributes of the recombinant protein obtained by transiently transfecting the eukaryotic cells is with +/−10% of the values of product quality attributes of the recombinant protein obtained by stable transfection. In some aspects, the quality attributes are selected from the group consisting of
(i) protein aggregation;
(ii) reduced and non-reduced species;
(iii) charge variants;
(iv) glycosylation profile; and,
(v) any combination thereof.

In some aspects, the protein aggregation quality attributes are selected from the group consisting of (i) percentage of high molecular weight species (HMW %), (ii) percentage of monomeric species, and (iii) any combination thereof. In some aspects, the protein aggregation quality attributes are determined using HPLC size exclusion chromatography. In some aspects, the reduced and non-reduced species quality attributes are selected from the group consisting of (i) percentage of reduced recombinant protein, (ii) percentage of non-reduced recombinant protein, and (iii) any combination thereof. In some aspects, the reduced and non-reduced species quality attributes are determined using capillary electrophoresis under reducing and non-reducing conditions (CE-SDS). In some aspects, the charge variants quality attributes are selected from the group consisting of (i) percentage of basic variants, (ii) percentage of acidic variant, (iii) percentage of main species, and (iv) any combination thereof. In some aspects, the charge variants quality attributes are determined by analyzing isoelectric distribution by capillary isoelectric focusing (iCIEF). In some aspects, the glycosylation profile comprises one or more N-linked glycans. In some aspects, the N-linked glycans comprise (mannose-3-N-acetylglucosamine-4-fucose) (G0F), mannose-3-N-acetylglucosamine-4-galactose-1-fucose (G1F), mannose-3-N-acetylglucosamine-4-galactose-2-fucose (G2F), mono-sialylated mannose-3-N-acetylglucosamine-4-galactose-1-fucose (S1G1F), mono-sialylated mannose-3-N-acetylglucosamine-4-galactose-2-fucose (S1G2F), mono-sialylated mannose-3-N-acetylglucosamine-4-galactose-3-fucose (S2G3F), di-sialylated mannose-3-N-acetylglucosamine-4-galactose-2-fucose (S2G2F), or any combination thereof. In some aspects, the glycosylation profile quality attributes are selected from the group consisting (i) percentage of G1F, (ii) percentage of G0F, (iii) percentage of G2F, (iv) percentage of total afucosylated protein, and (v) any combination thereof. In some aspects, the glycosylation profile quality attributes are determined using HPLC methods. In some aspects, the HPLC method is Ultra Performance Liquid Chromatography with fluorescence detection (UPLC-FLR).

The present disclosure also provides a recombinant protein obtained according to any of the methods disclosed above. In some aspects, the recombinant protein is antibody or antigen-binding fragment binds an antigen selected from the group consisting of PD-1, PD-L1, CVTLA-4, LAG-3, TIGIT, GITR, CXCR4, CD73, HER2, VEGF, CD20, CD40, CD11a, tissue factor (TF), PSCA, IL-8, EGFR, HER3, and HER4.

The present disclosure also provides a pharmaceutically composition comprising a recombinant protein obtained according to any of the methods disclosed above. Also provided is a cell or plurality thereof obtained according to any of the methods disclosed above. In some aspects, the cell or plurality of cells are Chinese hamster ovary (CHO) cells. The present disclosure also provides a bioreactor for the manufacture of a recombinant protein produced according to any of the methods disclosed above. Also provided is a bioreactor comprising the cell or plurality of cells disclosed above. The present disclosure also provides a system for high-yield production of a recombinant protein by transient transfection comprising (i) a bioreactor; (ii) an ATF perfusion system; and, (iii) an electroporation transfection system. In some aspects, the system is use to practice the method or to obtain the compositions (e.g., recombinant protein) disclosed above.

The present disclosure also provides a method to accelerate or shorten the development timeline of a recombinant protein comprising (i) culturing eukaryotic cells in a medium to concentrate the eukaryotic cells to a high density, and (ii) transiently transfecting the eukaryotic cells with a polynucleotide encoding the recombinant protein using electroporation. Also provides is a method to reduce the host cell toxicity of a recombinant protein comprising (i) culturing eukaryotic cells in a medium to concentrate the eukaryotic cells to a high density, and (ii) transiently transfecting the eukaryotic cells with a polynucleotide encoding the recombinant protein using electroporation. The present disclosure also provides a method to produce a recombinant protein for individualized therapeutics comprising (i) culturing eukaryotic cells in a medium to concentrate the eukaryotic cells to a high density, and (ii) transiently transfecting the eukaryotic cells with a polynucleotide encoding the recombinant protein using electroporation.

### BRIEF DESCRIPTION OF THE DRAWINGS/FIGURES

FIGS. 1A-1E show main effects plots for shake flask screening of key transient gene expression process parameters. FIG. 1A: Fold change (FC) titer for start density. FIG. 1B: Hour of application of temperature shift at 32° C. FIG. 1C: Addition of N, N-Dimethyl acetamide (DMA). FIG. 1D: Addition of Sodium Butyrate (NaBu). FIG. 1E: DNA: cell ratio (μg DNA/106 cells/mL). Error bars are expressed as one standard deviation.

FIGS. 2A-2D show bioreactor performance for N−1 seed perfusion and in-process attributes (N=4). FIG. 2A: Viable Cell Density (VCD, x 106 cells/mL) and viability determined by Trypan Blue Dye Exclusion using a Vi-CELL instrument. FIG. 2B: Glucose (g/L) levels.

FIG. 2C: Lactate (g/L) levels. FIG. 2D: Ammonium (mM) levels. All levels determined off-line via optical measurement using a BioProfile FLEX2™ instrument. Error bars are expressed as one standard deviation.

FIGS. 3A-3H show that continuous ATF perfusion post-concentration improves cell performance, cell replication pre-transfection and final titer. FIGS. 3A and 3E: Glucose concentration in N−1 seed perfusion post-concentration. FIGS. 3B and 3F: Distribution of G0/G1, S and G2/M phases in pre-transfected cells determined by Guava Cell Cycle Reagent. FIGS. 3C and 3G: Cell culture titer at 14 day. FIGS. 3D and 3G: Transfection efficiency determined at day 2 by direct staining of surface IgG using AF 488 anti-human IgG (H+L). Continuous perfusion (N=3) and discontinuous perfusion (N=1). Statistical difference was computed by t-test analysis and error bars are expressed as one standard deviation.

FIGS. 4A-4B show process development strategies of transient gene expression in AMBR® 250 bioreactor results in several reactor conditions with production titer >1 g/L. FIG. 4A: Main effects plots for each individual process parameter. FIG. 4B: Titer for top producing conditions. The common levels across the top conditions are highlighted.

FIGS. 5A-5E show successful scale-up of the transient gene expression process in 5 L reactors. FIG. 5A: Final titer in the AMBR® 250 A18 reactor (∘) and the scale-up process in 5 L reactor (□). FIG. 5B: Viable Cell Density (VCD, x 106 cells/mL) and viability determined by Trypan Blue Dye Exclusion using a Vi-CELL instrument. FIG. 5C: Lactate (g/L) levels. FIG. 5D: Ammonium (mM) levels. Lactate and ammonium levels determined off-line via optical measurement using a BioProfile FLEX2™ instrument. FIG. 5E: Lactate dehydrogenase (LDH) levels (U/L) determined off-line via CEDEX® Bio HT® analyzer.

FIGS. 6A-6D show that transient material is representative of stable material in terms of product quality attributes on day 14 at harvest for the fed-batch cell culture performance using AMBR® 250 (n=1) and 5-L (n=2) bioreactors for mAb production. FIG. 6A: Size exclusion chromatography (SEC). FIG. 6B: Capillarity electrophoresis under reduced and non-reduced conditions. FIG. 6C: Capillarity electrophoresis (ICE). FIG. 6D: N-glycosylation profiles.

FIG. 7 is a schematic representation comparing transient gene expression and stable cell line expression, showing that transient gene expression can rapidly generate drug substance material.

FIG. 8 is a schematic representation presenting potential uses of transient material generated according to the methods of the present disclosure to treat patients with unmet clinical needs.

FIG. 9 is a schematic representation of a representative work flow for transient gene expression as described herein. The workflow is capable of high protein yield and it is scalable.

### DETAILED DESCRIPTION

Traditional stable cell line development (CLD) to generate single clones typically takes between 3 and 5 months. Thus, transient expression of therapeutics is routinely used to produce small amounts of candidate proteins rapidly for preclinical research to shorten timelines and reduce overall costs. However, low productivity, inconsistent product quality of expressed protein and scalability issues have prevented the use of transient gene expression beyond non-GLP preclinical studies. The present disclosure presents a large-scale transient expression platform for rapid generation of proteins, e.g., monoclonal antibodies (mAbs) using flow-based electroporation technology at large scale (e.g., up to 5-L scale and above) using a eukaryotic cell platform, e.g., CHO cells. This platform can produce gram quantities of proteins with product quality profile comparable to the stable cell line generated protein. The present disclosure also describes the implementation of a tangential flow filtration system, e.g., an Alternating tangential flow filtration (ATF) system, to perform perfusion followed by concentration of the host cells to achieve high densities (e.g., 40×10⁶ to 200×10⁶ cells/ml densities, such as approximately 100×10⁶ cell/mL densities) and allow for flow-based electroporation. Extensive screening studies were conducted to optimize cell intensification, transfection, and process conditions to achieve high productivity and desired product quality profile. Overall process improvements have led transient titers as high as 2 g/L for model mAbs at up to 5 L scale, with quality of the transient recombinant product comparable that observed in recombinant product obtained from stably transfected host cells.

### Terms

In order that the present disclosure can be more readily understood, certain terms are first defined. As used in this application, except as otherwise expressly provided herein, each of the following terms shall have the meaning set forth below. Additional definitions are set forth throughout the application.

The singular forms “a”, “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

It is understood that wherever aspects are described herein with the language “comprising,” otherwise analogous aspects described in terms of “consisting of” and/or “consisting essentially of” are also provided.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As described herein, any concentration range, percentage range, ratio range or integer range is to be understood to include the value of any integer within the recited range and, when appropriate, fractions thereof (such as one tenth and one hundredth of an integer), unless otherwise indicated.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. The headings provided herein are not limitations of the various aspects of the disclosure, which can be had by reference to the specification as a whole. Accordingly, the terms defined are more fully defined by reference to the specification in its entirety.

Abbreviations used herein are defined throughout the present disclosure. Various aspects of the disclosure are described in further detail in the following subsections.

The terms “purifying,” “separating,” or “isolating,” as used interchangeably herein, refer to increasing the degree of purity of a protein of interest from a composition or sample comprising the protein of interest and one or more impurities. Typically, the degree of purity of the protein of interest is increased by removing (completely or partially) at least one impurity from the composition.

The terms “culture”, “cell culture” and “eukaryotic cell culture” as used herein refer to a cell population, either surface-attached or in suspension that is maintained or grown in a medium (see definition of “medium” below) under conditions suitable to survival and/or growth of the cell population. As will be clear to those of ordinary skill in the art, these terms as used herein can refer to the combination comprising the cell population and the medium in which the population is suspended.

The terms “media”, “medium”, “cell culture medium”, “culture medium”, “tissue culture medium”, “tissue culture media”, and “growth medium” as used herein refer to a solution containing nutrients which can be used to nourish growing cultured host cells. Typically, these solutions provide essential and non-essential amino acids, vitamins, energy sources, lipids, and trace elements required by the cell for minimal growth and/or survival. The solution can also contain components that enhance growth and/or survival above the minimal rate, including hormones and growth factors. The solution is formulated to a pH and salt concentration optimal for cell survival and proliferation. The medium can also be a “defined medium” or “chemically defined medium”—a serum-free medium that contains no proteins, hydrolysates or components of unknown composition. Defined media are free of animal-derived components and all components have a known chemical structure. One of skill in the art understands a defined medium can comprise recombinant glycoproteins or proteins, for example, but not limited to, hormones, cytokines, interleukins and other signaling molecules. Without limitation, these solutions ordinarily provide at least one component from one or more of the following categories: (1) an energy source, usually in the form of a carbohydrate such as glucose; (2) all essential amino acids, and usually the basic set of twenty amino acids plus cysteine; (3) vitamins and/or other organic compounds required at low concentrations; (4) free fatty acids or lipids, for example linoleic acid; and (5) trace elements, where trace elements are defined as inorganic compounds or naturally occurring elements that are typically required at very low concentrations, usually in the micromolar range. The nutrient solution can be supplemented electively with one or more components from any of the following categories: (1) hormones and other growth factors such as, serum, insulin, transferrin, and epidermal growth factor; (2) salts, for example, magnesium, calcium, and phosphate; (3) buffers, such as HEPES; (4) nucleosides and bases such as, adenosine, thymidine, and hypoxanthine; (5) protein and tissue hydrolysates, for example peptone or peptone mixtures which can be obtained from purified gelatin, plant material, or animal byproducts; (6) antibiotics, such as gentamycin; (7) cell protective agents, for example pluronic polyol; and (8) galactose. Commercially available media such as Ham's F10 (Sigma), Minimal Essential Medium ((MEM), (Sigma)), RPMI-1640 (Sigma), and Dulbecco's Modified Eagle's Medium ((DMEM), (Sigma)) are suitable for culturing the host cells. In addition, any of the media described in Ham et al., Meth. Enz. 58:44 (1979), Barnes et al., Anal. Biochem. 102:255 (1980) can be used as culture media for the host cells. Any other necessary supplements can also be included at appropriate concentrations.

As used herein, “culturing” refers to growing one or more cells in vitro under defined or controlled conditions. Examples of culturing conditions which can be defined include temperature, gas mixture, time, and medium formulation.

As used herein, “expanding” refers to culturing one or more cells in vitro for the purpose of obtaining a larger number of cells in the culture.

As used herein, the terms “expression” or “expresses” are used to refer to transcription and translation occurring within a cell. The level of expression of a product gene in a host cell can be determined on the basis of either the amount of corresponding mRNA that is present in the cell or the amount of the protein encoded by the product gene that is produced by the cell, or both.

The term “cell viability” as used herein refers to the ability of cells in culture to survive under a given set of culture conditions or experimental variations. The term as used herein also refers to that portion of cells which are alive at a particular time in relation to the total number of cells, living and dead, in the culture at that time.

In some aspects, the medium comprises glucose at a concentration sufficient to maintain a high cell density. In some aspects, the concentration of glucose in the medium is at least about 0.1 g/L, at least about 0.5 g/L, at least about 1.0 g/L, at least about 1.5 g/L, at least about 2.0 g/L, at least about 2.5 g/L, at least about 3.0 g/L, at least about 3.5 g/L, at least about 4.0 g/L, at least about 4.5 g/L, at least about 5.0 g/L, at least about 6.0 g/L, at least about 7.0 g/L, at least about 8.0 g/L, at least about 9.0 g/L, or at least about 10.0 g/L.

In some aspects, the concentration of glucose in the medium is about 0.1 g/L, about 0.5 g/L, about 1.0 g/L, about 1.5 g/L, about 2.0 g/L, about 2.5 g/L, about 3.0 g/L, about 3.5 g/L, about 4.0 g/L, about 4.5 g/L, about 5.0 g/L, about 6.0 g/L, about 7.0 g/L, about 8.0 g/L, about 9.0 g/L, or about 10.0 g/L.

As used herein, the terms “reference method,” “reference process,” or “baseline condition,” refer to a process or method of producing the same recombinant protein which is identical to a methods of the present disclosure, except for the specific conditions used for the methods of the present disclosure to increase protein yield using transient expression. For example, a reference process or reference method can be a process that does not include (i) transient transfection via electroporation, e.g., flow electroporation, (ii) cell concentration using tangential flow filtration, e.g., alternating tangential flow filtration, (iii) perfusion cell culture, e.g., continuous perfusion, or (iv) a combination thereof. A reference method can be used as a baseline (“baseline condition”) to which the methods of the present disclosure are compared. In some aspects, the values of the parameters (e.g., protein yield) measured after applying method of the present disclosure can be normalized with respect to a baseline condition.

The term “antibody” refers, in some aspects, to a protein comprising at least two heavy (H) chains and two light (L) chains inter-connected by disulfide bonds. Each heavy chain is comprised of a heavy chain variable region (abbreviated herein as VH) and a heavy chain constant region (abbreviated herein as CH). In some antibodies, e.g., naturally-occurring IgG antibodies, the heavy chain constant region is comprised of a hinge and three domains, CH1, CH2 and CH3. In some antibodies, e.g., naturally-occurring IgG antibodies, each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The term “antibody” can include a bispecific antibody or a multispecific antibody.

An “IgG antibody”, e.g., a human IgG1, IgG2, IgG3 and IgG4 antibody, as used herein has, in some aspects, the structure of a naturally-occurring IgG antibody, i.e., it has the same number of heavy and light chains and disulfide bonds as a naturally-occurring IgG antibody of the same subclass. For example, an IgG1, IgG2, IgG3 or IgG4 antibody may consist of two heavy chains (HCs) and two light chains (LCs), wherein the two HCs and LCs are linked by the same number and location of disulfide bridges that occur in naturally-occurring IgG1, IgG2, IgG3 and IgG4 antibodies, respectively (unless the antibody has been mutated to modify the disulfide bridges).

An immunoglobulin can be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. Immunoglobulins, e.g., IgG1, exist in several allotypes, which differ from each other in at most a few amino acids. “Antibody” includes, by way of example, both naturally-occurring and non-naturally-occurring antibodies; monoclonal and polyclonal antibodies; chimeric and humanized antibodies; human and nonhuman antibodies and wholly synthetic antibodies.

The term “antigen-binding portion” of an antibody, as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment (fragment from papain cleavage) or a similar monovalent fragment consisting of the VL, VH, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; (vi) an isolated complementarity determining region (CDR) and (vii) a combination of two or more isolated CDRs which can optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences.

As used herein, “isotype” refers to the antibody class (e.g., IgG1, IgG2, IgG3, IgG4, IgM, IgA1, IgA2, IgD, and IgE antibody) that is encoded by the heavy chain constant region genes.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter codes.

As used herein, the term “polypeptide” refers to a molecule composed of monomers (amino acids) linearly linked by amide bonds (also known as peptide bonds). The terms “polypeptide” or “protein” or “product” or “product protein” or “amino acid residue sequence” are used interchangeably. The term “polypeptide” refers to any chain or chains of two or more amino acids, and does not refer to a specific length of the product. As used herein the term “protein” is intended to encompass a molecule comprised of one or more polypeptides, which can in some instances be associated by bonds other than amide bonds. On the other hand, a protein can also be a single polypeptide chain. In this latter instance the single polypeptide chain can in some instances comprise two or more polypeptide subunits fused together to form a protein. The terms “polypeptide” and “protein” also refer to the products of post-expression modifications, including without limitation glycosylation, acetylation, phosphorylation, amidation, derivatization by known protecting/blocking groups, proteolytic cleavage, or modification by non-naturally occurring amino acids. A polypeptide or protein can be derived from a natural biological source or produced by recombinant technology.

The terms “polynucleotide” or “nucleotide” as used herein are intended to encompass a singular nucleic acid as well as plural nucleic acids, and refers to an isolated nucleic acid molecule or construct, e.g., messenger RNA (mRNA), complementary DNA (cDNA), or plasmid DNA (pDNA). The term “nucleic acid” refers to any one or more nucleic acid segments, e.g., DNA, cDNA, or RNA fragments, present in a polynucleotide. When applied to a nucleic acid or polynucleotide, the term “isolated” refers to a nucleic acid molecule, DNA or RNA, which has been removed from its native environment, for example, a recombinant polynucleotide encoding an antigen binding protein contained in a vector is considered isolated for the purposes of the present disclosure. Further examples of an isolated polynucleotide include recombinant polynucleotides maintained in heterologous host cells or purified (partially or substantially) from other polynucleotides in a solution. Isolated RNA molecules include in vivo or in vitro RNA transcripts of polynucleotides of the present disclosure. Isolated polynucleotides or nucleic acids according to the present disclosure further include such molecules produced synthetically. In addition, a polynucleotide or a nucleic acid can include regulatory elements such as promoters, enhancers, ribosome binding sites, or transcription termination signals.

### I. Methods for Large Scale Transient Expression of Therapeutic Proteins

The present disclosure provides methods for high-yield production of recombinant proteins, e.g., therapeutic antibodies, in a bioreactor (e.g., a high-throughput bioreactor or a large scale bioreactor) comprising
(i) culturing eukaryotic cells in a medium to concentrate the eukaryotic cells to a high density, and
(ii) transiently transfecting the eukaryotic cells with a polynucleotide encoding the recombinant protein using electroporation. In some aspects, the method further comprises
(iii) post-transfection culturing the transiently transfected eukaryotic cells.

The present disclosure also provides methods for high quality production of recombinant proteins, e.g., therapeutic antibodies, in a bioreactor (e.g., a high-throughput bioreactor or a large scale bioreactor) comprising
(i) culturing eukaryotic cells in a medium to concentrate the eukaryotic cells to a high density, and
(ii) transiently transfecting the eukaryotic cells with a polynucleotide encoding the recombinant protein using electroporation. In some aspects, the method further comprises post-transfection culturing the transiently transfected eukaryotic cells.

Also provided methods for high yield and/or high quality production of recombinant proteins, e.g., therapeutic antibodies, in a bioreactor (e.g., a high-throughput bioreactor or a large scale bioreactor) comprising
(i) culturing eukaryotic cells in a medium to concentrate the eukaryotic cells to a high density, and
(ii) transiently transfecting the eukaryotic cells with a polynucleotide encoding the recombinant protein using electroporation. In some aspects, the method further comprises post-transfection culturing the transiently transfected eukaryotic cells.

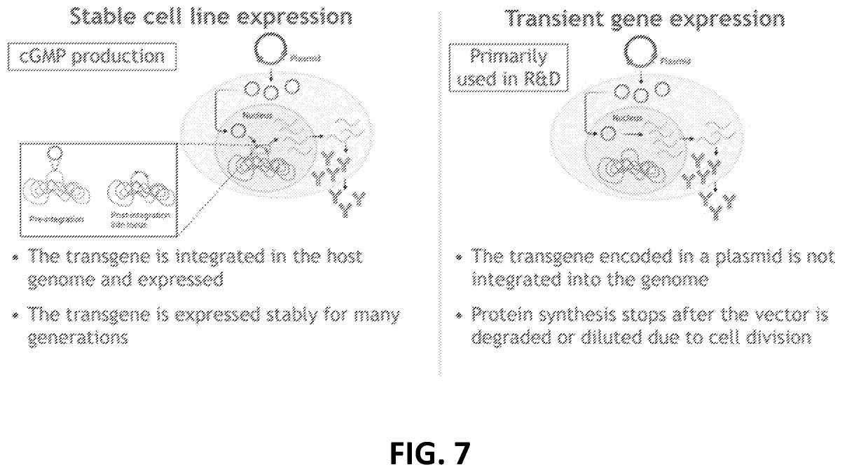

The method for transient gene expression disclosed herein can rapidly generate drug substance material (see FIG. 7). Whereas in stable cell line expression (generally used for cGMP production) the transgene is integrated in the host genome and expressed stably for many generations, in transient gene expression (primarily used in r&D), the transgene is encoded in a plasmid instead of being integrated in the genome and protein synthesis stops after the vector is degraded or diluted due to cell division. The methods disclosed herein allow the use of transient gene expression beyond R&D, for example, to accelerate timelines for cGMP production. Most importantly, the transient technology disclosed herein can generate representative material comparable to the material produced by stable cell lines.

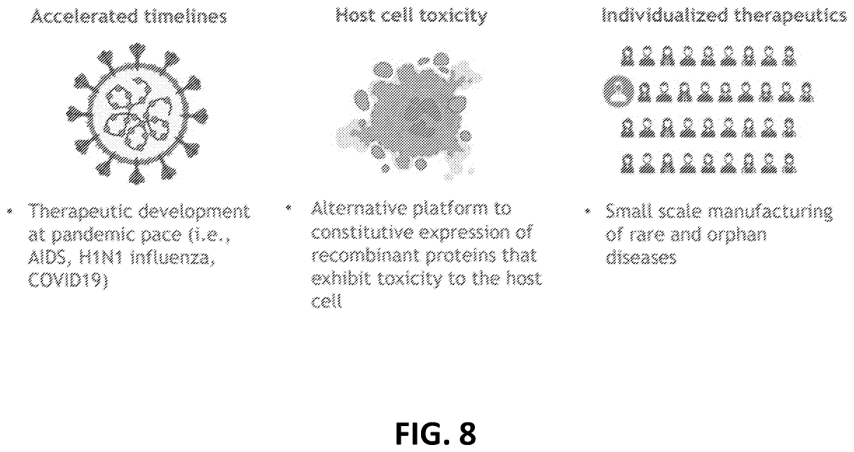

These methods for production of high quality material using transient gene expression can be used for example, (i) to accelerate development timelines, (ii) to circumvent host cell toxicity, or (iii) to develop individualized therapeutics (FIG. 8). The possibility of accelerating development timelines can further therapeutic development at pandemic pace, for example, to generate drug products to treat AIDS, H1N1 influenza, or COVID19. In some instance, recombinant products can be toxic to host cells. The transient gene methods disclosed herein can provide alternative platforms to constitutive expression of recombinant proteins that exhibit toxicity to the host cell by reducing the time that cells are exposed to the toxic recombinant product. Another use of the transient gene expression disclosed herein is the development of individualized therapeutics. This is possible by allowing the production of high quality drug products to treat rare and orphan via small scale manufacturing pipelines.

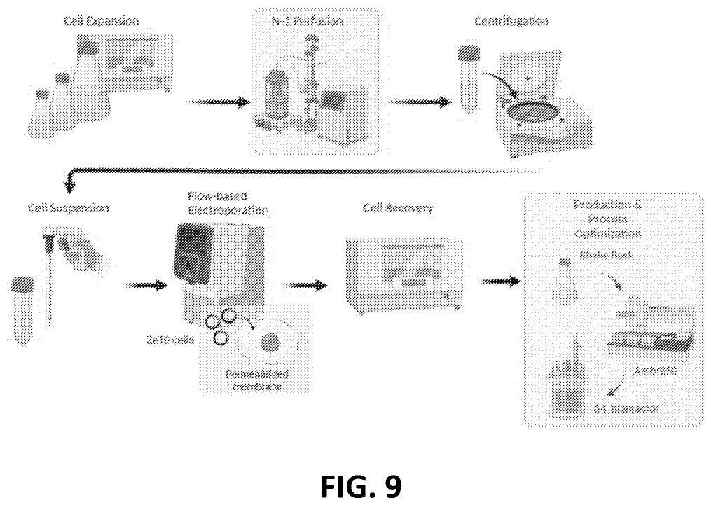

A transient gene expression workflow that could be used for the uses outlined above is shown in FIG. 9. The workflow shown in FIG. 9 can be scaled, e.g., by
(i) substituting the perfusion system with a larger system or running several equivalent systems in parallel,
(ii) replacing the flow-based electroporation system with a larger system or running several equivalent systems in parallel,
(iii) increasing the scale of culture vessels following cell recovery, or
(iv) combinations thereof.

Increasing the scale of culture vessels following cell recovery can comprise substituting shaker flasks with larger productions vessels such as high-throughput reactors (e.g., AMBR™500) or liter scale bioreactors. In some aspects, the culture vessel scale-up can proceed to bioreactors with volumes of 10 L, 50 L, 100 L, or larger. In some aspects, the workflow can be scaled-up by using larger volume high-throughput bioreactors or multiple high-throughput bioreactors in series. Likewise, in place of a larger bioreactor (e.g., a 10 L bioreactor), smaller bioreactors with an equivalent total volume may be used (e.g., two 5 L bioreactors).

The present disclosure provides a method to accelerate or shorten the development timeline of a recombinant protein, e.g., a therapeutic antibody, comprising
(i) culturing eukaryotic cells in a medium to concentrate the eukaryotic cells to a high density, and
(ii) transiently transfecting the eukaryotic cells with a polynucleotide encoding the recombinant protein using electroporation.

In some aspects, the method further comprises post-transfection culturing the transiently transfected eukaryotic cells. In some aspects, the development is shortened by at least about 10%, at least about 20%, at least about 30%, at least about 40%, at least about 50%, at least about 60%, or at least about 70% compared to a normal development timeline.

The present disclosure provides a method to reduce the host cell toxicity of a recombinant protein, e.g., a therapeutic antibody, comprising
(i) culturing eukaryotic cells in a medium to concentrate the eukaryotic cells to a high density, and
(ii) transiently transfecting the eukaryotic cells with a polynucleotide encoding the recombinant protein using electroporation.

In some aspects, the method further comprises post-transfection culturing the transiently transfected eukaryotic cells. In some aspects, the cell host toxicity is reduced by at least about 10%, at least about 20%, at least about 30%, at least about 40%, at least about 50%, at least about 60%, at least about 70%, at least about 80%, or at least about 90% compared to the host cell toxicity observed in stably transfected cell lines.

The present disclosure provides a method to produce a recombinant protein, e.g., a therapeutic antibody, for individualized therapeutics comprising
(i) culturing eukaryotic cells in a medium to concentrate the eukaryotic cells to a high density, and
(ii) transiently transfecting the eukaryotic cells with a polynucleotide encoding the recombinant protein using electroporation.

In some aspects, the method further comprises post-transfection culturing the transiently transfected eukaryotic cells. In some aspects, the recombinant protein, e.g., a therapeutic antibody, for individualized therapeutics is a therapeutic agent to treat a rare disease. In some aspects, the recombinant protein, e.g., a therapeutic antibody, for individualized therapeutics is a therapeutic agent to treat an orphan disease. In some aspects, the recombinant protein, e.g., a therapeutic antibody, for individualized therapeutics is a therapeutic agent for personalized medicine.

In some aspects, the cells are concentrated to a high density prior to transient transfection while maintaining them in optimal growth phase.

Post-transfection culturing of the transiently transfected eukaryotic cells according to the present disclosure leads to the production of recombinant proteins (transient transfection material) with product quality comparable to the clonal material.

As used herein, the term “transient transfection material” refers to a recombinant protein or combination thereof that is obtained through a production process resulting from the use of transiently transfected cells as described in the present disclosure.

As used herein, the term “clonal material” refers to a recombinant protein or combination thereof, that is obtained through a production process comprising the use of cells stably transfected. In some aspects, the clonal material is produced by a process comprising upstream (cell culture conditions, cell culture media, etc.) and downstream (e.g., harvesting, filtrations steps, chromatographic steps) steps that are identical or substantially identical to the steps used to produce a transient material, except that the cells have been stably transfected.

As used herein the term “product quality comparable to the clonal material” refers to a transient transfection material having the quality attributes, e.g., selected from the group consisting of (i) protein aggregation; (ii) reduced and non-reduced species; (iii) charge variants; (iv) glycosylation profile; and, (v) any combination, that differ no more than about 1%, about 2%, about 3%, about 4%, about 5%, about 5%, about 6%, about 7%, about 8%, about 9%, about 10%, about 11%, about 12%, about 13%, about 14%, about 15%, about 16%, about 1%, about 18%, about 19% or about 20% with respect to the value of the corresponding attribute or combination thereof determined for clonal material.

As used herein the term “optimal growth phase” refers to the growth of cells in culture so that a majority of the cells is in replication phases (S and G2/M). In some aspects, at least about 50%, at least about 55%, at least about 60%, at least about 65%, at least about 70%, at least about 75%, at least about 80%, or at least about 85% of the cells are in the replicating phases. In some aspect, between about 60% and about 80% of the cells are in the replicating phases. In some aspects, between about 65% and 75% of the cells are in the replicating phases. In some aspects, about 50% of the cells are in the replicating phases. In some aspects, about 60% of the cells are in the replicating phases. In some aspects, about 70% of the cells are in the replicating phases. In some aspects, about 75% of the cells are in the replicating phases. In some aspects, about 80% of the cells are in the replicating phases.

In some aspects, the medium in (i) comprises glucose at a concentration sufficient to maintain a high cell density. In some aspects, the concentration of glucose in the medium is at least about 0.1 g/L, at least about 0.5 g/L, at least about 1.0 g/L, at least about 1.5 g/L, at least about 2.0 g/L, at least about 2.5 g/L, at least about 3.0 g/L, at least about 3.5 g/L, at least about 4.0 g/L, at least about 4.5 g/L, or at least about 5.0 g/L. In some aspects, the concentration of glucose in the medium is between about 2 g/L and about 6 g/L, 2 g/L, 3 g/L, 4 g/L, 5 g/L or 6 g/L.

The methods of the present disclosure can be performed in a variety of vessel types or bioreactor to accommodate various recombinant protein production strategies. In some aspects, the bioreactor is a high-throughput bioreactor, e.g., an AMBR®250 bioreactor or AMBR®15 bioreactor. In some aspects, the high-throughput bioreaction is a system for parallel fermentation. In some aspects, each bioreactor in the high-throughput bioreactor has a volume between 100 mL and 250 mL, e.g., 100 mL, 150 mL, 200 mL, or 250 mL. In some aspects, the high-throughput bioreactor comprises multiple individual bioreactors, e.g., 12 or 24 bioreactors. In some aspects, the individual bioreactors are single-use bioreactors. In some aspects. The high-throughput bioreactor is a fully automated system controlling, e.g., media fill, inoculation, sampling, feeding, temperature, impeller speed, pH, optical density (OD), or any combination thereof.

In some aspects, the bioreactor, e.g., a high-throughput bioreactor such as an AMBR®250 bioreactor comprises at least one integrated cell culture analyzer. In some aspects, the cell culture analyzer is a pH measurement module, e.g., an AMBR® Analysis module. In some aspects, the cell culture analyzer is an integrated cell counter, e.g., a Beckman Vicell XR cell counter or a Cedex HiRes cell counter. In some aspects, the cell culture analyzer comprises a Nova Biomedical FLEX2 External Sampling Module (ESM), and a FLEX2 Analyzer, or a combination thereof. In some aspects, automated sample, sample transfer and analysis, data transfer and automated feedback control are conducted by a cell culture analyzer, e.g., a FLEX2 Analyzer. In some aspects, control actions can be executed by the cell culture analyzer, e.g., a FLEX2 Analyzer such as glucose or feed addition base on glucose level, cell count, viable cell count, or other parameters.

In some aspects, the bioreactor is a large scale bioreactor. As used herein, the term “large scale bioreactor” refers to a bioreactor having a volume of 1 L, 2 L, 5 L, 10 L, or higher, e.g., about 20 L, about 25 L, about 50 L, about 100 L about 500 L, about 1000 L, about 5000 L, about 10,000 L, or about 20,000 L. In some aspects, “large scale bioreactor” means a volume of at least 1000 L, e.g., 1000 L to 10,000 L, e.g., 5000 L. In some aspects, the term “large scale bioreactor” means a volume of at least 2000 L.

The methods of the present disclosure can involve a fed-batch culture. Fed-batch culture is a method of culturing cells in which additional components are provided to the culture at some time subsequent to the beginning of the culture process. A fed-batch culture can be started using a basal medium. The culture medium with which additional components are provided to the culture at some time subsequent to the beginning of the culture process is a feed medium. A fed-batch culture is typically stopped at some point and the cells and/or components in the medium are harvested and optionally purified. Accordingly, in some aspects of the present disclosure, the bioreactor is a fed-batch production bioreactor. In some aspects, the bioreactor is a single use bioreactor. In some aspects, the bioreactor is a glass tank bioreactor.

The methods of the present disclosure can involve a perfusion culture. Perfusion culture involves a continuous flow of a physiological nutrient solution at a steady rate, through or over a population of cells. As perfusion systems generally involve the retention of the cells within the culture unit, perfusion cultures characteristically have relatively high cell densities, but the culture conditions are difficult to maintain and control. In addition, since the cells are grown to and then retained within the culture unit at high densities, the growth rate typically continuously decreases over time, leading to the late exponential or even stationary phase of cell growth.

As used herein “perfusion” or “perfusion culture” or “perfusion reactor process” refers to flow of a physiological nutrient solution at a steady rate, through or over a population of cells. As perfusion systems generally involve the retention of the cells within the culture unit, perfusion cultures characteristically have relatively high cell densities, but the culture conditions are difficult to maintain and control. In addition, since the cells are grown to and then retained within the culture unit at high densities, the growth rate typically continuously decreases over time, leading to the late exponential or even stationary phase of cell growth. This culture strategy generally comprises culturing mammalian cells, e.g., non-anchorage dependent cells, expressing a polypeptide and/or virus of interest during a production phase in a continuous cell culture system. In some aspects, perfusion can be “continuous perfusion.”

The term “continuous perfusion” refers to a steady state cell culture system or process that operates without interruption and in which cells or micro-organisms are maintained in culture by the continuous addition of fresh medium. The term “continuous,” means uninterrupted in time, sequence and/or operation for prolonged periods of time. As used in reference to the processes of the present disclosure, “continuous” means that the processes are physically and logistically integrated so as to permit operation without interruption for a prolonged period of time. In other aspects, perfusion can be “discontinuous perfusion.”

By “non-anchorage dependent cells” is meant cells propagating freely in suspension throughout the bulk of a culture, as opposed to being attached or fixed to a solid substrate during propagation. The continuous cell culture system can comprise a cell retention device similar to that used in a perfusion system, but that allows continuous removal of a significant portion of the cells, such that a smaller percentage of the cells are retained than in perfusion culture.

By “cell retention device” is meant any structure capable of retaining cells, particularly non-anchorage dependent cells, in a particular location during cell culture. Non-limiting examples include microcarriers, fine mesh spin filters, hollow fibers, flat plate membrane filters, settling tubes, ultrasonic cell retention devices, and the like, that can retain non-anchorage dependent cells within bioreactors. Polypeptides and/or viruses of interest (e.g., a recombinant polypeptide and/or recombinant virus) can be recovered from the cell culture system, e.g., from medium removed from the cell culture system.

In some aspects, the perfusion cell culture is conducted using tangential flow filtration. In some aspect of the present disclosure, tangential flow filtration is also used to concentrate cells to a high density, for example, prior to transfection.

The term “tangential flow filtration” or “TFF”, also known as cross-flow filtration, refers to a specific filtration method in which a solute-containing solution passes tangentially (tangential flow) across an ultrafiltration membrane and lower molecular weight solutes are passed through a filter surface, e.g., the surface of a filtering membrane, by applying pressure. Tangential flow filtration is used in concentration processes, for example, to increase the concentration of cells in a medium such as a cell culture medium. As used herein, “tangential flow” refers to a flow substantially parallel to the filter surface, for example, unidirectional tangential flow (TFF) or cross-flow.

In specific aspects, the tangential flow filtration is alternating tangential flow filtration (ATF). The term “alternating tangential flow” as used herein refers to a flow arrangement in which a tangential flow travels back and forth along the membrane surface of a hollow-fiber filter and another flow travels in a direction substantially perpendicular to said filter surface. A tangential flow or an alternating tangential flow can be achieved according to methods known to the person skilled in the art. For example, U.S. Pat. No. 6,544,424 describes methods for generating an alternating tangential flow in hollow fiber filters.

The higher molecular weight solute-containing solution passing tangentially across the ultrafiltration membrane is retained, and thus this solution is referred to herein as “retentate.” The lower molecular weight solutes that pass through the ultrafiltration membrane are referred to herein as “permeate.” Thus, the retentate is concentrated by flowing along, e.g., tangentially, the surface of an ultrafiltration membrane under pressure. The ultrafiltration membrane has pore size with a certain cut off value. In some aspects, the cutoff value is about 50 kDa or less, e.g., 50 kDa, 40 kDa, 30 kDa, 20 kDa, or 10 Da. In some aspects, the cutoff value is 30 kD or less.

In one aspect, the system used to perform the methods disclosed herein comprises a fluid filtration system comprising a cell culture device including at least one filter containing housing, a fluid connector for directing the cell culture fluid from a cell culture vessel (bioreactor) to the cell culture device, at least one pump which powers the fluid in one or alternating direction(s) through the filter containing housing, and at least one fluid harvest port. The system is useful for conducting a rapid, low sheer, tangential flow filtration. Such a system has applications in perfusion cell culture systems or any other culture systems which requires retaining viable cells within the bioreactor.

In some aspects, perfusion culture and cell concentrations are conducted using a single-use device, for example, a REPLIGEN™ ATF system. In some aspects, the REPLIGEN™ ATF system is an Xcell ATF® 1 system (0.5 L to 2 L suspension culture volume), Xcell ATF® 2 system (2 L to 10 L suspension culture volume), Xcell ATF® 4 system (10 L to 50 L suspension culture volume), Xcell ATF® 6 system (50 L to 200 L suspension culture volume), or Xcell ATF® 10 system (200 L to 1000 L suspension culture volume). In some aspects, the REPLIGEN™ ATF system is connected to an XCell™ Lab Controller or XCell™ C410 Controller. Similar devices known in the art can be used to implement the methods disclosed herein, e.g., an EMD Millipore PROSTAK™ TFF system.

As used herein, the term “high density” when used in the context of culturing cells in accordance with the present disclosure, and of methods of the disclosure employing same for the purpose of conducting transfection workflows, generally refers to a known cell line, or a variant of a known cell line, that can be grown or cultured in an appropriate cell culture medium to densities of greater than, e.g., about 40×10⁶ cells/mL, or, e.g., between about 40×10⁶ cells/mL and about 200×10⁶ cells/mL. In some specific aspects, high density can refer to above 200×10⁶ cells/mL.

In some aspects of the methods disclosed herein, the cells are concentrated using a tangential flow filtration method such as ATF to a density of at least about 40×10⁶ cells/mL, at least about 50×10⁶ cells/mL, at least about 60×10⁶ cells/mL, at least about 70×10⁶ cells/mL, at least about 80×10⁶ cells/mL, at least about 90×10⁶ cells/mL, at least about 100×10⁶ cells/mL, at least about 110×10⁶ cells/mL, at least about 120×10⁶ cells/mL, at least about 130×10⁶ cells/mL, at least about 140×10⁶ cells/mL, at least about 150×10⁶ cells/mL, at least about 160×10⁶ cells/mL, at least about 170×10⁶ cells/mL, at least about 180×10⁶ cells/mL, at least about 190×10⁶ cells/mL, or at least about 200×10⁶ cells/mL. In some aspects of the methods disclosed herein, the cells are concentrated using a tangential flow filtration method such as ATF to a density of about 40×10⁶ cells/mL, about 50×10⁶ cells/mL, about 60×10⁶ cells/mL, about 70×10⁶ cells/mL, about 80×10⁶ cells/mL, about 90×10⁶ cells/mL, about 100×10⁶ cells/mL, about 110×10⁶ cells/mL, about 120×10⁶ cells/mL, about 130×10⁶ cells/mL, about 140×10⁶ cells/mL, about 150×10⁶ cells/mL, about 160×10⁶ cells/mL, about 170×10⁶ cells/mL, about 180×10⁶ cells/mL, about 190×10⁶ cells/mL, about 200×10⁶ cells/mL, about 210×10⁶ cells/mL, about 220×10⁶ cells/mL, about 230×10⁶ cells/mL, about 240×10⁶ cells/mL, or about 250×10⁶ cells/mL. In some aspects, the cells are concentrated using a tangential flow filtration method such as ATF to a density of about 40×10⁶ cells/mL. In some aspects, the cells are concentrated using a tangential flow filtration method such as ATF to a density of about 50×10⁶ cells/mL. In some aspects, the cells are concentrated using a tangential flow filtration method such as ATF to a density of about 60×10⁶ cells/mL In some aspects, the cells are concentrated using a tangential flow filtration method such as ATF to a density of about 70×10⁶ cells/mL. In some aspects, the cells are concentrated using a tangential flow filtration method such as ATF to a density of about 80×10⁶ cells/mL. In some aspects, the cells are concentrated using a tangential flow filtration method such as ATF to a density of about 90×10⁶ cells/mL. In some aspects, the cells are concentrated using a tangential flow filtration method such as ATF to a density of about 100×10⁶ cells/mL. In some aspects, the cells are concentrated using a tangential flow filtration method such as ATF to a density of about 110×10⁶ cells/mL. In some aspects, the cells are concentrated using a tangential flow filtration method such as ATF to a density of about 120×10⁶ cells/mL. In some aspects, the cells are concentrated using a tangential flow filtration method such as ATF to a density of about 130×10⁶ cells/mL. In some aspects, the cells are concentrated using a tangential flow filtration method such as ATF to a density of about 140×10⁶ cells/mL. In some aspects, the cells are concentrated using a tangential flow filtration method such as ATF to a density of about 150×10⁶. In some aspects, the cells are concentrated using a tangential flow filtration method such as ATF to a density of about 160×10⁶ cells/mL. In some aspects, the cells are concentrated using a tangential flow filtration method such as ATF to a density of about 170×10⁶ cells/mL. In some aspects, the cells are concentrated using a tangential flow filtration method such as ATF to a density of about 180×10⁶ cells/mL. In some aspects, the cells are concentrated using a tangential flow filtration method such as ATF to a density of about 190×10⁶ cells/mL. In some aspects, the cells are concentrated using a tangential flow filtration method such as ATF to a density of about 200×10⁶ cells/mL. In some aspects, the cells are concentrated using a tangential flow filtration method such as ATF to a density of about 210×10⁶ cells/mL. In some aspects, the cells are concentrated using a tangential flow filtration method such as ATF to a density of about 220×10⁶ cells/mL. In some aspects, the cells are concentrated using a tangential flow filtration method such as ATF to a density of about 230×10⁶ cells/mL. In some aspects, the cells are concentrated using a tangential flow filtration method such as ATF to a density of about 240×10⁶ cells/mL. In some aspects, the cells are concentrated using a tangential flow filtration method such as ATF to a density of about 250×10⁶ cells/mL.

In some aspects cells/mL, the cells are concentrated using a tangential flow filtration method such as ATF to a density of about 40×10⁶ cells/mL to about 200×10⁶ cells/mL, about 60×10⁶ cells/mL to about 200×10⁶ cells/mL, about 80×10⁶ cells/mL to about 200×10⁶ cells/mL, about 100×10⁶ cells/mL to about 200×10⁶ cells/mL, about 120×10⁶ cells/mL to about 200×10⁶ cells/mL, about 140×10⁶ cells/mL to about 200×10⁶ cells/mL, about 160×10⁶ cells/mL to about 200×10⁶ cells/mL, about 180×10⁶ cells/mL to about 200×10⁶ cells/mL, about 40×10⁶ cells/mL to about 180×10⁶ cells/mL, about 60×10⁶ cells/mL to about 180×10⁶ cells/mL, about 80×10⁶ cells/mL to about 180×10⁶ cells/mL, about 100×10⁶ cells/mL to about 180×10⁶ cells/mL, about 120×10⁶ cells/mL to about 180×10⁶ cells/mL, about 140×10⁶ cells/mL to about 180×10⁶ cells/mL, about 160×10⁶ cells/mL to about 180×10⁶ cells/mL, about 40×10⁶ cells/mL to about 160×10⁶ cells/mL, about 60×10⁶ cells/mL to about 160×10⁶ cells/mL, about 80×10⁶ cells/mL to about 160×10⁶ cells/mL, about 100×10⁶ cells/mL to about 160×10⁶ cells/mL, about 120×10⁶ cells/mL to about 160×10⁶ cells/mL, about 140×10⁶ cells/mL to about 160×10⁶ cells/mL, about 40×10⁶ cells/mL to about 140×10⁶ cells/mL, about 60×10⁶ cells/mL to about 140×10⁶ cells/mL, about 80×10⁶ cells/mL to about 140×10⁶ cells/mL, about 100×10⁶ cells/mL to about 140×10⁶ cells/mL, about 120×10⁶ cells/mL to about 140×10⁶ cells/mL, about 40×10⁶ cells/mL to about 120×10⁶ cells/mL, about 60×10⁶ cells/mL to about 120×10⁶ cells/mL, about 80×10⁶ cells/mL to about 120×10⁶ cells/mL, about 100×10⁶ cells/mL to about 120×10⁶ cells/mL, about 40×10⁶ cells/mL to about 100×10⁶ cells/mL, about 60×10⁶ cells/mL to about 100×10⁶ cells/mL, about 80×10⁶ cells/mL to about 100×10⁶ cells/mL.

As used herein, the term “transfection” refers to the delivery of an exogenous nucleic acid (e.g., a polynucleotide encoding the recombinant protein) to a target cell or host cell, such that the nucleic acid is expressed in the cell. The term “transient transfection” refers to a process in which the nucleic acid introduced into a cell is not required to integrate into the genome or chromosomal DNA of that cell. It is in fact predominantly maintained as an extrachromosomal element, e.g., as an episome, in the cell. Transcription processes of the nucleic acid of the episome are not affected and, e.g., a protein encoded by the nucleic acid of the episome is produced. The term “transiently transfected cells” refers to a cell comprising an exogenous nucleic acid that is not stably integrated into the genome/chromosome of a host cell. The exogenous nucleic acid may be only temporarily-heritable or non-heritable. An example of transient transfection is a vector that is transfected into the cell, but does not chromosomally integrate, and may further require selective pressure in order to maintain it in the cell host for a period of time (for example 1-5, or 2-3 days). The term “transient expression” refers to expression of an exogenous nucleic acid (e.g., a polynucleotide encoding the recombinant protein) to yield a recombinant protein in a transiently transfected cell of the present disclosure.

The methods of the present disclosure comprise the use of electroporation to facilitate the entry of one or more nucleic acid molecules into host cells. As used herein, the term “electroporation” refers to application of an electrical current or electrical field to a cell to facilitate entry of a nucleic acid molecule into the cell. One of skill in the art would understand that any method and technique of electroporation is contemplated by the present invention. In some aspects, cells are electroporated using flow electroporation. The term “flow electroporation” refers to electroporation of cells within a fluid chamber flow path. This method is particularly effective for large scale volume of cells. Static electroporation, by contrast, involves electroporation of a set and limited volume of cells due to constraints associated with moving electricity across liquid and the distance between opposing electrodes.

Two main advantages of the use of flow electroporation are the speed at which a large population of cells can be transfected and the ability to overcome thermal degradation of the sample. In some aspects, electroporation is conducted using a MAXCYTE™ transfection system.

In some aspects, flow electroporation can be performed using MAXCYTE™ STX®, MAXCYTE™ VLS® or MAXCYTE™ GT® flow electroporation instrumentation. In some specific aspects, the transfection system is a MAXCYTE™ STX® transfection system. In some specific aspects, the transfection system is a MAXCYTE™ VLX® transfection system.

The methods of transfecting cells by electroporation disclosed herein, e.g., using flow electroporation, are capable of achieving transfection efficiencies of greater than 40%, greater than 50%, greater than 60%, greater than 70%, greater than 80%, greater than 80%, or greater than 90%. Transfection efficiency can be measured either by the percentage of cells that express the product of the gene or the secretion levels of the product expressed by the gene. The cells maintain a high viability during and after the electroporation process. Viability of electroporated cells can be at least about 50%, at least about 55%, at least about 60%, at least about 65%, at least about 70%, at least about 75%, at least about 80%, at least about 85%, at least about 90%, at least about 95%, or at least about 97% of the viability of the starting, unelectroporated population of host cells or an electroporated population of host cells transfected with a control polynucleotide.

Flow electroporation involves, e.g., transferring a suspension of cells and loading molecules into an apparatus comprised of a fluid chamber or fluid flow path; the said fluid chamber or fluid flow path being comprised of electrodes disposed along sides of the fluid chamber or fluid flow path and configured to subject biological particles within the fluid chamber fluid flow path to an electric field suitable for electroporation; and transferring the electroporated cell suspension out of the apparatus. In some aspects, the methods disclosed herein comprise using a flow electroporation apparatus for electrical stimulation of suspensions of particles, comprising a flow electroporation cell assembly having one or more inlet flow portals, one or more outlet flow portals, and one or more flow channels, the flow channels being comprised of two or more walls, with the flow channels further being configured to receive and transiently contain a continuous flow of particles in suspension from the inlet flow portals; and paired electrodes disposed in relation to the flow channels such that each electrode forms at least one wall of the flow channels, the electrodes further comprising placing the electrodes in electrical communication with a source of electrical energy, whereby suspensions of particles flowing through the channels may be subjected to an electrical field formed between the electrodes.

The process is initiated by attaching the flow cell with solutions and cell suspensions in the containers with the necessary fluids and samples. Priming solution (saline) and cell suspension are introduced by providing the required commands to the electroporation system, which controls operation of the pump and pinch valves. As the cells transit the flow path between electrodes, electric pulses of the chosen voltage, duration, and frequency are applied. Product and waste fluids are collected in the designated containers. The user inputs the desired voltage and other parameters into the flow electroporation system of the present invention. A range of settings is optionally available. The computer communicates to the electronics in the tower to charge the capacitor bank to the desired voltage. Appropriate switches then manipulate the voltage before it is delivered to the flow path to create the electric field (the switches provide alternating pulses or bursts to minimize electrode wear brought on by prolonged exposure to the electric field). The voltage is delivered according to the duration and frequency parameters set into the flow electroporation system of the present invention by the operator.

The flow electroporation process can be initiated by, for example, placing an electroporation chamber in fluid communication with solutions and cell suspensions in containers (e.g., via tubing), which may be carried out in an aseptic or sterile environment. A cell suspension and/or other reagents may be introduced to the electroporation chamber using one or more pumps, vacuums, valves, other mechanical devices that change the air pressure or volume inside the electroporation chamber and combinations thereof, which can cause the cell suspension and/or other reagents to flow into the electroporation chamber at a desired time and at the desired rate. If a portion of the cell suspension and/or other reagents is positioned in the electroporation chamber, electric pulses of a desired voltage, duration, and/or interval are applied the cell suspension and/or other reagents. After electroporation, the processed cell suspension and/or other reagents can be removed from the electroporation chamber using one or more pumps, vacuums, valves, other electrical, mechanical, pneumatic, or microfluidic devices that change the displacement, pressure or volume inside the electroporation chamber, and combinations thereof. In certain embodiments, gravity or manual transfer may be used to move sample or processed sample into or out of an electroporation chamber. If desired, a new cell suspension and/or other reagents can be introduced into the electroporation chamber. An electroporated sample can be collected separately from a sample that has not yet been electroporated. The preceding series of events can be coordinated temporally by a computer coupled to, for example, electronic circuitry (e.g., that provides the electrical pulse), pumps, vacuums, valves, combinations thereof, and other components that effect and control the flow of a sample into and out of the electroporation chamber. As an example, the electroporation process can be implemented by a computer, including by an operator through a graphic user interface on a monitor and/or a keyboard. Examples of suitable valves include pinch valves, butterfly valves, and/or ball valves. Examples of suitable pumps include centrifugal or positive displacement pumps.

In some aspects, electroporation, e.g., flow electroporation, is conducted at a DNA to cell ratio of between about 1 μg DNA per 1×10⁶ cells to about 2 μg DNA per 1×10⁶ cells. In some aspects, electroporation, e.g., flow electroporation, is conducted at a DNA to cell ratio of about 1.5 μg DNA per 1×10⁶ cells. In some aspects, electroporation, e.g., flow electroporation, is conducted at a DNA to cell ratio of about 1 μg DNA per 1×10⁶ cells, about 1.1 μg DNA per 1×10⁶ cells, about 1.2 μg DNA per 1×10⁶ cells, about 1.3 μg DNA per 1×10⁶ cells, about 1.4 μg DNA per 1×10⁶ cells, about 1.5 μg DNA per 1×10⁶ cells, about 1.6 μg DNA per 1×10⁶ cells, about 1.7 μg DNA per 1×10⁶ cells, about 1.8 μg DNA per 1×10⁶ cells, about 1.9 μg DNA per 1×10⁶ cells, or about 2 μg DNA per 1×10⁶ cells. In some aspects, electroporation, e.g., flow electroporation, is conducted at a DNA to cell ratio of about 1 μg DNA per 1×10⁶ cells. In some aspects, electroporation, e.g., flow electroporation, is conducted at a DNA to cell ratio of about 1.1 μg DNA per 1×10⁶ cells. In some aspects, electroporation, e.g., flow electroporation, is conducted at a DNA to cell ratio of about 1.2 μg DNA per 1×10⁶ cells. In some aspects, electroporation, e.g., flow electroporation, is conducted at a DNA to cell ratio of about 1.3 μg DNA per 1×10⁶ cells. In some aspects, electroporation, e.g., flow electroporation, is conducted at a DNA to cell ratio of about 1.4 μg DNA per 1×10⁶ cells. In some aspects, electroporation, e.g., flow electroporation, is conducted at a DNA to cell ratio of about 1.5 μg DNA per 1×10⁶ cells. In some aspects, electroporation, e.g., flow electroporation, is conducted at a DNA to cell ratio of about 1.6 μg DNA per 1×10⁶ cells. In some aspects, electroporation, e.g., flow electroporation, is conducted at a DNA to cell ratio of about 1.7 μg DNA per 1×10⁶ cells. In some aspects, electroporation, e.g., flow electroporation, is conducted at a DNA to cell ratio of about 1.8 μg DNA per 1×10⁶ cells. In some aspects, electroporation, e.g., flow electroporation, is conducted at a DNA to cell ratio of about 1.9 μg DNA per 1×10⁶ cells. In some aspects, electroporation, e.g., flow electroporation, is conducted at a DNA to cell ratio of about 2 μg DNA per 1×10⁶ cells.

In some aspects, electroporation, e.g., flow electroporation, is conducted at a cell density of about 40×10⁶ cells/mL, about 50×10⁶ cells/mL, about 60×10⁶ cells/mL, about 70×10⁶ cells/mL, about 80×10⁶ cells/mL, about 90×10⁶ cells/mL, about 100×10⁶ cells/mL, about 110×10⁶ cells/mL, about 120×10⁶ cells/mL, about 130×10⁶ cells/mL, about 140×10⁶ cells/mL, about 150×10⁶ cells/mL, about 160×10⁶ cells/mL, about 170×10⁶ cells/mL, about 180×10⁶ cells/mL, about 190×10⁶ cells/mL, about 200×10⁶ cells/mL, about 210×10⁶ cells/mL, about 220×10⁶ cells/mL, about 230×10⁶ cells/mL, about 240×10⁶ cells/mL, or about 250×10⁶ cells/mL. In some aspects, electroporation, e.g., flow electroporation, is conducted at a cell density of at least about 40×10⁶ cells/mL, at least about 50×10⁶ cells/mL, at least about 60×10⁶ cells/mL, at least about 70×10⁶ cells/mL, at least about 80×10⁶ cells/mL, at least about 90×10⁶ cells/mL, at least about 100×10⁶ cells/mL, at least about 110×10⁶ cells/mL, at least about 120×10⁶ cells/mL, at least about 130×10⁶ cells/mL, at least about 140×10⁶ cells/mL, at least about 150×10⁶ cells/mL, at least about 160×10⁶ cells/mL, at least about 170×10⁶ cells/mL, at least about 180×10⁶ cells/mL, at least about 190×10⁶ cells/mL, at least about 200×10⁶ cells/mL, about 210×10⁶ cells/mL, about 220×10⁶ cells/mL, about 230×10⁶ cells/mL, about 240×10⁶ cells/mL, or about 250×10⁶ cells/mL. In some aspects, electroporation, e.g., flow electroporation, is conducted at a cell density of between about 40×10⁶ cells/mL and about 60×10⁶ cells/mL, between about 60×10⁶ cells/mL and about 80×10⁶ cells/mL, between about 80×10⁶ cells/mL and about 100×10⁶ cells/mL, between about 100×10⁶ cells/mL and about 120×10⁶ cells/mL, between about 120×10⁶ cells/mL and about 140×10⁶ cells/mL, between about 140×10⁶ cells/mL and about 160×10⁶ cells/mL, between about 160×10⁶ cells/mL and about 180×10⁶ cells/mL, between about 180×10⁶ cells/mL and about 200×10⁶ cells/mL, between about 50×10⁶ cells/mL and about 75×10⁶ cells/mL, between about 75×10⁶ cells/mL and about 100×10⁶ cells/mL, between about 100×10⁶ cells/mL and about 125×10⁶ cells/mL, between about 125×10⁶ cells/mL and about 150×10⁶ cells/mL, between about 150×10⁶ cells/mL and about 175×10⁶ cells/mL, between about 175×10⁶ cells/mL and about 200×10⁶ cells/mL, between about 100×10⁶ cells/mL and about 150×10⁶ cells/mL, between about 150×10⁶ cells/mL and about 200×10⁶ cells/mL, between about 100×10⁶ cells/mL and about 200×10⁶ cells/mL or between about 200×10⁶ cells/mL and about 250×10⁶. In some aspects, electroporation, e.g., flow electroporation, is conducted at a cell density of about 40×10⁶ cells/mL. In some aspects, electroporation, e.g., flow electroporation, is conducted at a cell density of about 50×10⁶ cells/mL. In some aspects, electroporation, e.g., flow electroporation, is conducted at a cell density of about 60×10⁶ cells/mL. In some aspects, electroporation, e.g., flow electroporation, is conducted at a cell density of about 70×10⁶ cells/mL. In some aspects, electroporation, e.g., flow electroporation, is conducted at a cell density of about 80×10⁶ cells/mL. In some aspects, electroporation, e.g., flow electroporation, is conducted at a cell density of about 90×10⁶ cells/mL. In some aspects, electroporation, e.g., flow electroporation, is conducted at a cell density of about 100×10⁶ cells/mL. In some aspects, electroporation, e.g., flow electroporation, is conducted at a cell density of about 110×10⁶ cells/mL. In some aspects, electroporation, e.g., flow electroporation, is conducted at a cell density of about 120×10⁶ cells/mL. In some aspects, electroporation, e.g., flow electroporation, is conducted at a cell density of about 130×10⁶ cells/mL. In some aspects, electroporation, e.g., flow electroporation, is conducted at a cell density of about 140×10⁶ cells/mL. In some aspects, electroporation, e.g., flow electroporation, is conducted at a cell density of about 150×10⁶ cells/mL. In some aspects, electroporation, e.g., flow electroporation, is conducted at a cell density of about 160×10⁶ cells/mL. In some aspects, electroporation, e.g., flow electroporation, is conducted at a cell density of about 170×10⁶ cells/mL. In some aspects, electroporation, e.g., flow electroporation, is conducted at a cell density of about 180×10⁶ cells/mL. In some aspects, electroporation, e.g., flow electroporation, is conducted at a cell density of about 190×10⁶ cells/mL. In some aspects, electroporation, e.g., flow electroporation, is conducted at a cell density of about 200×10⁶ cells/mL. In some aspects, electroporation, e.g., flow electroporation, is conducted at a cell density of about 210×10⁶ cells/mL. In some aspects, electroporation, e.g., flow electroporation, is conducted at a cell density of about 220×10⁶ cells/mL. In some aspects, electroporation, e.g., flow electroporation, is conducted at a cell density of about 230×10⁶ cells/mL. In some aspects, electroporation, e.g., flow electroporation, is conducted at a cell density of about 240×10⁶ cells/mL. In some aspects, electroporation, e.g., flow electroporation, is conducted at a cell density of about 250×10⁶ cells/mL.

The eukaryotic cells used in the methods disclosed herein can be transfected with expression or cloning vectors for producing recombinant proteins and cultured as appropriate. In some aspects, standard molecular biology techniques are used to prepare the recombinant expression vectors, culture the cells, and recover the product from the culture medium. In some aspects, the cell culture media described herein can be used as culture media for hybridoma cells, monoclonal antibody producing cells, virus-producing cells, transfected cells, cancer cells and/or recombinant peptide producing cells.

An important aspects of the methods disclosed herein is the tuning of the perfusion reactor post concentration and transfection. In some aspects, the perfusion system, e.g., an Xcell ATF® 2 system, is adjusted to replenish glucose levels. In some aspects, the perfusion system, e.g., an Xcell ATF® 2 system, is adjusted to replenish glucose levels, up to approximately 4 g/L. In some aspects, the perfusion system, e.g., an Xcell ATF® 2 system, is adjusted to replenish glucose levels to a level sufficient to prevent cells to transition to a quiescent state. In some aspects, the perfusion system, e.g., an Xcell ATF® 2 system, is adjusted so at least 60% of cells are in the replication phases (S and G2/M). In some aspects, the perfusion system, e.g., an Xcell ATF® 2 system, is adjusted so at least about 60%, at least about 65%, at least about 70%, or at least about 75% of cells are in the replication phases (S and G2/M). In some aspects, the use of continuous ATF perfusion can result is an increase in titer compared to batch mode about three-fold.

The cells of the present disclosure can be cultured under suitable conditions for suitable periods of time after transfection, and under conditions that depend on the type(s) of cells being cultured and the product being produced. In some aspects, the cells are cultured post-transfection for about two to about fourteen days. In some aspects, the cells are cultured from about four to about ten days. In some aspects, the cells are cultured for about two days, about three days, about four days, about five days, about six days, about seven days, about eight days, about nine days, about 10 days, about 11 days, about 12 days, about 13 days, about 14 days, about 15 days, about 16 days, about 17 days, about 18 days, about 19 days, about 20 days, or about 21 days. In some aspects, the cells are cultured for at least about two days, at least about three days, at least about four days, at least about five days, at least about six days, at least about seven days, at least about eight days, at least about nine days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, or at least about 21 days. In some aspects, the cells are culture from about 2 day to about 5 days, from about 3 days to about 7 days, from about 4 days to about 10 days, from about 5 days to about 10 days, from about 2 days to about 7 days, from about 10 days to about 15 days, from about 15 days to about 20 days, from about 7 days to about 14 days, from about 7 days to about 21 days, or from about 14 days to about 21 days. In some aspects, the cells are cultured for about 7 days. In some aspects, the cells are cultured for about 14 days. In some aspects, the cells are cultured for about 21 days.

In some aspects, the post-transfection cell culture start density is between 5×10⁶ and 15×10⁶ cells/mL. In some aspects, the post-transfection cell culture start density is about 5×10⁶ cells/mL, about 6×10⁶ cells/mL, about 7×10⁶ cells/mL, about 8×10⁶ cells/mL, about 9×10⁶ cells/mL, about 10×10⁶ cells/mL, about 11×10⁶ cells/mL, about 12×10⁶ cells/mL, about 13×10⁶ cells/mL, about 14×10⁶ cells/mL, about 15×10⁶ cells/mL, about 16×10⁶ cells/mL, about 17×10⁶ cells/mL, about 18×10⁶ cells/mL, about 19×10⁶ cells/mL, or about 20×10⁶ cells/mL. In some aspects, the post-transfection cell culture start density is at least about 5×10⁶ cells/mL, at least about 6×10⁶ cells/mL, at least about 7×10⁶ cells/mL, at least about 8×10⁶ cells/mL, at least about 9×10⁶ cells/mL, at least about 10×10⁶ cells/mL, at least about 11×10⁶ cells/mL, at least about 12×10⁶ cells/mL, at least about 13×10⁶ cells/mL, at least about 14×10⁶ cells/mL, at least about 15×10⁶ cells/mL, at least about 16×10⁶ cells/mL, at least about 17×10⁶ cells/mL, at least about 18×10⁶ cells/mL, at least about 19×10⁶ cells/mL, or at least about 20×10⁶ cells/mL. In some aspects, the post-transfection cell culture start density is between about 5×10⁶ cells/mL and about 10×10⁶ cells/mL, between about 10×10⁶ cells/mL and about 15×10⁶ cells/mL, between about 15×10⁶ cells/mL and about 20×10⁶ cells/mL, between about 5×10⁶ cells/mL and about 15×10⁶ cells/mL, or between about 10×10⁶ cells/mL and about 20×10⁶ cells/mL. In some aspects, the post-transfection cell culture start density is about 5×10⁶ cells/mL. In some aspects, the post-transfection cell culture start density is about 6×10⁶ cells/mL. In some aspects, the post-transfection cell culture start density is about 7×10⁶ cells/mL. In some aspects, the post-transfection cell culture start density is about 8×10⁶ cells/mL. In some aspects, the post-transfection cell culture start density is about 9×10⁶ cells/mL. In some aspects, the post-transfection cell culture start density is about 10×10⁶ cells/mL. In some aspects, the post-transfection cell culture start density is about 11×10⁶ cells/mL. In some aspects, the post-transfection cell culture start density is about 12×10⁶ cells/mL. In some aspects, the post-transfection cell culture start density is about 13×10⁶ cells/mL. In some aspects, the post-transfection cell culture start density is about 14×10⁶ cells/mL In some aspects, the post-transfection cell culture start density is about 15×10⁶ cells/mL. In some aspects, the post-transfection cell culture start density is about 16×10⁶ cells/mL. In some aspects, the post-transfection cell culture start density is about 17×10⁶ cells/mL. In some aspects, the post-transfection cell culture start density is about 18×10⁶ cells/mL. In some aspects, the post-transfection cell culture start density is about 19×10⁶ cells/mL. In some aspects, the post-transfection cell culture start density is about 20×10⁶ cells/mL.

In some aspects, the post-transfection cell culture is shifted temperature for some of the culture period. In some aspects, the temperature of the post-transfection cell culture is shifted for some of the culture period. The temperature of a production vessel such as a bioreactor can be an important aspect of bioproduction because the temperature of the bioreactor plays a role in cell growth, viable cell density, cell longevity and/or glycosylation activity of glycosylating enzymes inside a cell. Temperature changes can significantly affect the rate of enzymatic reactions within the cell, denature proteins, and/or cause other effects on a cell culture. Cells can be cultured at an initial temperature set point such as 37° C., for example, to encourage maximum viable cell density, and then the temperature can be modified to another temperature set point (i.e., a second temperature set point or a final temperature set point) to prolong cell longevity or to enhance desired glycosylation activity within the cell. In some aspects, the methods disclosed herein comprise one or more temperature adjustments during protein production. In some aspects, temperature adjustments can be a decrease of operating temperature during the manufacturing process.

In some aspects, the post-transfection cell culture comprises a temperature shift on Day 1 or Day 2. In some aspects, the initial temperature set point for the present method is about 37° C. and a second temperature set point is lower than about 36° C., e.g., about 35° C., about 34° C., about 33° C., about 32° C., or about 31° C.

In some aspects, the initial temperature set point is about 37° C. and a second temperature set point is lower than about 34° C. In some aspects, the initial temperature set point is about 36° C. and a second temperature set point is lower than about 35° C., about 34° C., or about 33° C. In some aspects, the initial temperature set point is lower than about 36.5° C. and the final temperature set point is about 31° C. In some aspects, the initial temperature set point is about 36.0° C. and the final temperature set point is about 31° C. In some aspects, the initial temperature set point is lower than about 35.5° C. and the final temperature set point is about 31° C. In some aspects, the initial temperature set point is lower than about 35.0° C. and the final temperature set point is about 31° C. In some aspects, the initial temperature set point is lower than about 36.5° C., a second temperature set point is about 33° C., and a final temperature set point is lower than about 33° C. or about 32° C. In some aspects, the initial temperature set point is about 36.0° C., a second temperature set point is about 33° C., and a final temperature set point is lower than about 33° C. or about 32° C. In some aspects, the initial temperature set point is about 36.0° C., a second temperature set point is about 33° C., and a final temperature set point is lower than about 32° C. In some aspects, the initial temperature set point is about 36.0° C., a second temperature set point is about 33° C., and a final temperature set point is about 31° C. In some aspects, the temperature shift is from about 37° C. to about 32° C. In some aspects, the temperature shift is from about 37° C. to about 34° C.

In some aspects, the second temperature set point occurs at about 1 day (Day 1), 2 days (Day 2), 3 days (Day 3), 4 days (Day 4), 5 days (Day 5), about 6 days (Day 6), or about 7 days (Day 7), after the initial temperature set point. In some aspects, the temperature shift is from about 37° C. to about 32° C. on Day 1. In some aspects, the temperature shift is from about 37° C. to about 32° C. on Day 2. In other aspects, the temperature shift is from about 37° C. to about 34° C. on Day 1. In some aspects, the temperature shift is from about 37° C. to about 32° C. on Day 2. In other aspects, the temperature shift is from about 37° C. to about 34° C. on Day 2.

In some aspects, the post-transfection cell culture comprises the addition of N,N-dimethyl acetamide (DMA) to the culture medium. In some aspects, DMA is added to the culture medium at a concentration between about 0.125% v/v and about 0.250% v/v. In some aspects, DMA is added to the culture medium at a concentration of about 0.125% v/v. In some aspects, DMA is added to the culture medium at a concentration of about 0.250% v/v. In some DMA is added to the culture medium at a concentration of about 0.05% v/v, about 0.1% v/v, about 0.15% v/v, about 0.2% v/v, about 0.25% v/v, about 0.3% v/v, about 0.35% v/v, about 0.4% v/v, about 0.45% v/v, or about 0.5% v/v. In some DMA is added to the culture medium at a concentration of about 0.05% v/v. In some DMA is added to the culture medium at a concentration of about 0.1% v/v. In some DMA is added to the culture medium at a concentration of about 0.15% v/v. In some DMA is added to the culture medium at a concentration of about 0.2% v/v. In some DMA is added to the culture medium at a concentration of about 0.25% v/v. In some DMA is added to the culture medium at a concentration of about 0.3% v/v. In some DMA is added to the culture medium at a concentration of about 0.35% v/v. In some DMA is added to the culture medium at a concentration of about 0.4% v/v. In some DMA is added to the culture medium at a concentration of about 0.45% v/v. In some DMA is added to the culture medium at a concentration of about 0.5% v/v. In some DMA is added to the culture medium at a concentration between about 0.05% v/v and about 0.1% v/v, between about 0.1% v/v and about 0.15% v/v, between about 0.15% v/v and about 0.2% v/v, between about 0.2% v/v and about 0.25% v/v, between about 0.25% v/v and about 0.3% v/v, between about 0.3% v/v and about 0.35% v/v, between about 0.35% v/v and about 0.4% v/v, between about 0.4% v/v and about 0.45% v/v, between about 0.45% v/v and about 0.5% v/v.

In some aspects, the post-transfection cell culture comprises the addition of sodium butyrate (NaBu.) to the culture medium. In some aspects, NaBu is added to the culture medium at a concentration between about 1 mM and about 2 mM. In some aspects, NaBu is added to the culture medium at a concentration of about 1 mM. In some aspects, NaBu is added to the culture medium at a concentration of about 2 mM. In some aspects, NaBu is added to the culture medium at a concentration of about 0.1 mM to about 5 mM. In some aspects, NaBu is added to the culture medium at a concentration of about 0.1 mM, about 0.25 mM, about 0.5 mM, about 1 mM, about 1.5 mM, about 2 mM, about 2.5 mM, about 3 mM, about 3.5 mM, about 4 mM, about 4.5 mM, or about 5 mM. In some aspects, NaBu is added to the culture medium at a concentration between about 0.1 mM and about 0.25 mM, between about 0.25 mM and about 0.5 mM, between about 0.5 mM and about 1 mM, between about 1 mM and about 1.5 mM, between about 1.5 mM and about 2 mM, between about 2 mM and about 2.5 mM, between about 2.5 mM and about 3 mM, between about 3 mM and about 3.5 mM, between about 3.5 mM and about 4 mM, between about 4 mM and about 4.5 mM, or between about 4.5 mM and about 5 mM.

The methods disclosed herein can produce high yields of recombinant proteins when using transient transfection. As used herein, the term “high yield” relates to the production of between 0.2 and 1.5 g/L, of recombinant protein per liter of cell culture medium. In some aspects, high yield is at least 1 g/L. High yield can also be defined as a yield comparable to that obtained using clonal cells. In some aspects, high yield is a yield above that obtained using clonal cells, for example, a yield about 10%, about 20%, about 30%, about 40%, about 50%, about 60%, about 70%, about 80%, about 90%, about 100%, or higher compared to the yield obtained when stably transfected cells are used. The methods disclosed herein can also produce recombinant proteins with high quality when using transient transfection. As disclosed above, high quality proteins obtained according to the methods disclosed herein are proteins with quality attributes related, e.g., to (i) protein aggregation (e.g., percentage of high molecular weight species (HMW %) and/or or percentage of monomeric species); (ii) reduced and non-reduced species (e.g., percentage of reduced recombinant protein and/or percentage of non-reduced recombinant protein); (iii) charge variants (e.g., percentage of basic variants, percentage of acidic variant, or percentage of main species); (iv) glycosylation profile (e.g., percentage of G1F, G0F, G2F, total afucosylated protein, or any combination) which have a product quality comparable to clonal material (e.g., quality attributes or combinations thereof with values that are, e.g., plus/minus 5%, 10%, or 20% with respect the corresponding values obtained from clonal material).

In some aspects, the recombinant protein yield of the methods disclosed herein (e.g., wherein the recombinant protein comprises an antibody or antigen-binding portion thereof) is at least about 1 g/L, at least about 2 g/L, at least about 3 g/L, at least about 4 g/L, at least about 5 g/L, at least about 6 g/L, at least about 7 g/L, at least about 8 g/L, at least about 9 g/L or at least about 10 g/L. In some aspects, the recombinant protein yield of the methods disclosed herein (e.g., wherein the recombinant protein comprises an antibody or antigen-binding portion thereof) is about 1 g/L, about 2 g/L, about 3 g/L, about 4 g/L, about 5 g/L, about 6 g/L, about 7 g/L, about 8 g/L, about 9 g/L or about 10 g/L. In some aspects, the yield of recombinant mAb produced according to the methods disclosed herein is about 1 g/L. In some aspects, the yield of recombinant mAb produced according to the methods disclosed herein is about 2 g/L. In some aspects, the yield of recombinant mAb produced according to the methods disclosed herein is about 3 g/L. In some aspects, the yield of recombinant mAb produced according to the methods disclosed herein is about 4 g/L. In some aspects, the yield of recombinant mAb produced according to the methods disclosed herein is about 5 g/L. In some aspects, the yield of recombinant mAb produced according to the methods disclosed herein is about 6 g/L. In some aspects, the yield of recombinant mAb produced according to the methods disclosed herein is about 7 g/L. In some aspects, the yield of recombinant mAb produced according to the methods disclosed herein is about 8 g/L. In some aspects, the yield of recombinant mAb produced according to the methods disclosed herein is about 9 g/L. In some aspects, the yield of recombinant mAb produced according to the methods disclosed herein is about 10 g/L. In some aspects, these high yields can be achieved after 7 days of culture. In some aspects, these high yields can be achieved after 14 days of culture. In some aspects, the recombinant protein yield of the methods disclosed herein (e.g., wherein the recombinant protein comprises an antibody or antigen-binding portion thereof) is about 1 g/L after 7 days of culture. In some aspects, the recombinant protein yield of the methods disclosed herein (e.g., wherein the recombinant protein comprises an antibody or antigen-binding portion thereof) is about 2 g/L after 14 days of culture.

The present disclosure provides a method for high-yield production of a recombinant protein (e.g., antibody such as a mAb or an antigen-binding portion thereof) in a bioreactor (e.g., an high-throughput bioreactor such as an AMBR® 250 bioreactor or a large-volume bioreactor such as a 5 L bioreactor) comprising:
(i) culturing eukaryotic cells (e.g., CHO cells) under continuous ATF perfusion (e.g., using a REPLIGEN™ ATF system such as an Xcell ATF® 2 system);
(ii) concentrating the cells to a density between 40 and 200×10⁶ cells/mL (e.g., 160×10⁶ cell/mL to 200×10⁶ cells/mL) using continuous ATF perfusion (e.g., using a REPLIGEN™ ATF system such as an Xcell ATF® 2 system);
(iii) transiently transfecting the eukaryotic cells with a polynucleotide encoding the recombinant protein (e.g., antibody such as a mAb or an antigen-binding portion thereof) using flow-based electroporation (e.g., using a MAXCYTE™ STX® transfection system, and, e.g., a DNA to cell ratio of about 1.5 μg DNA per 1×10⁶ cells); and.
(iv) post-transfection culturing the transiently transfected eukaryotic cells in a bioreactor (e.g., an high-throughput bioreactor such as an AMBR® 250 bioreactor or a large-volume bioreactor such as a 5 L bioreactor).

In some aspects, post-transfection culturing comprises
(i) a cell culture start density of about 5×10⁶ cells/mL to 15×10⁶ cells/mL,
(ii) optionally, DMA at about 0.125% (v/v/),
(iii) NaBu at about 1 mM, and
(iv) a temperature shift at Day 1 from about 36.5° C. to about 32° C., wherein the volume of the bioreactor is between about 250 mL and about 5 L.

In some aspects, post-transfection culturing comprises
(i) a cell culture start density of about 15×10⁶ cells/mL±10%,
(ii) optionally, DMA at about 0.125% (v/v/)±10%,
(iii) NaBu at about 1 mM±10%, and
(iv) a temperature shift at Day 1 or Day 2 from about 36° C.±1° C. to about 32° C.±1° C., wherein the volume of the bioreactor is between about 250 mL and about 5 L.

In some aspects, post-transfection culturing comprises
(i) a cell culture start density of about 15×10⁶ cells/mL±10%,
(ii) optionally, DMA at about 0.125% (v/v/)±10%,
(iii) NaBu at about 1 mM±10%, and
(iv) a temperature shift at Day 1 or Day 2 from about 36° C.±1° C. to about 32° C.±1° C., wherein the volume of the bioreactor is between about 250 mL and about 5 L.

In some aspects, post-transfection culturing comprises
(i) a cell culture start density of about 15×10⁶ cells/mL±10%,
(ii) optionally, DMA at about 0.125% (v/v/)±10%,
(iii) NaBu at about 1 mM±10%, and
(iv) a temperature shift at Day 1 from about 37° C.±1° C. to about 32° C.±2° C., wherein the volume of the bioreactor is about 250 mL.

In some aspects, post-transfection culturing comprises
(i) a cell culture start density of about 15×10⁶ cells/mL±10%,
(ii) optionally, DMA at about 0.125% (v/v/)±10%,
(iii) NaBu at about 1 mM±10%, and
(iv) a temperature shift at Day 1 from about 37° C.±1° C. to about 32° C.±2° C., wherein the volume of the bioreactor is about 5 L.

In some aspects, post-transfection culturing comprises
(i) a cell culture start density of about 15×10⁶ cells/mL±10%,
(ii) optionally, DMA at about 0.125% (v/v/)±10%,
(iii) NaBu at about 1 mM±10%, and
(iv) a temperature shift at Day 1 from about 37° C. to about 32° C., wherein the volume of the bioreactor is about 250 mL,
wherein the basal media is formulated for intensified cell culture manufacturing and culture comprises a P/V shift.

In some aspects, post-transfection culturing comprises
(i) a cell culture start density of about 15×10⁶ cells/mL±10%,
(ii) optionally, DMA at about 0.125% (v/v/)±10%,
(iii) NaBu at about 1 mM±10%, and
(iv) a temperature shift at Day 1 from about 37° C. to about 32° C., wherein the volume of the bioreactor is about 250 mL,
wherein the culture comprises a P/V shift.

In some aspects, post-transfection culturing comprises
(i) a cell culture start density of about 5×10⁶ cells/mL±10%,
(ii) optionally, DMA at about 0.125% (v/v/)±10%,
(iii) NaBu at about 1 mM±10%, and
(iv) a temperature shift at Day 1 from about 37° C. to about 32° C., wherein the volume of the bioreactor is about 250 mL,
wherein the basal medium is formulated for intensified cell culture manufacturing and culture does not comprise a P/V shift.

In some aspects, post-transfection culturing comprises
(i) a cell culture start density of about 15×10⁶ cells/mL±10%,
(ii) optionally, DMA at about 0.125% (v/v/)±10%, and
(iii) a temperature shift at Day 2 from about 37° C. to about 32° C., wherein the volume of the bioreactor is about 250 mL,
wherein the basal medium is formulated for intensified cell culture manufacturing and culture does not comprise a P/V shift.

In some aspects, post-transfection culturing comprises
(i) a cell culture start density of about 15×10⁶ cells/mL±10%,
(ii) optionally, DMA at about 0.125% (v/v/)±10%,
(iii) NaBu at about 1 mM±10%, and
(iv) a temperature shift at Day 2 from about 37° C. to about 34° C., wherein the volume of the bioreactor is about 250 mL,
wherein the basal medium is formulated for intensified cell culture manufacturing and culture comprises a P/V shift.

In some aspects, post-transfection culturing comprises
(i) a cell culture start density of about 15×10⁶ cells/mL±10%,
(ii) optionally, DMA at about 0.125% (v/v/)±10%, and
(iii) a temperature shift at Day 1 from about 37° C. to about 32° C., wherein the volume of the bioreactor is about 250 mL,
wherein the culture comprises a P/V shift.

In some aspects, the methods disclosed herein improve the recombinant protein yield by at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, or at least about 400% as compared to a reference method without the optimized culturing conditions disclosed herein.

In some aspects, the methods disclosed herein improve the protein yield by at least about 100% compared to a reference method. In some aspects, the methods disclosed herein improve the protein yield by at least about 110% compared to a reference method. In some aspects, the methods disclosed herein improve the protein yield by at least about 120% compared to a reference method. In some aspects, the methods disclosed herein improve the protein yield by at least about 130% compared to a reference method. In some aspects, the methods disclosed herein improve the protein yield by at least about 140% compared to a reference method. In some aspects, the methods disclosed herein improve the protein yield by at least about 150% compared to a reference method. In some aspects, the methods disclosed herein improve the protein yield by at least about 160% compared to a reference method. In some aspects, the methods disclosed herein improve the protein yield by at least about 170% compared to a reference method. In some aspects, the methods disclosed herein improve the protein yield by at least about 180% compared to a reference method. In some aspects, the methods disclosed herein improve the protein yield by at least about 190% compared to a reference method. In some aspects, the methods disclosed herein improve the protein yield by at least about 200% compared to a reference method. In some aspects, the methods disclosed herein improve the protein yield by at least about 210% compared to a reference method. In some aspects, the methods disclosed herein improve the protein yield by at least about 220% compared to a reference method. In some aspects, the methods disclosed herein improve the protein yield by at least about 230% compared to a reference method. In some aspects, the methods disclosed herein improve the protein yield by at least about 240% compared to a reference method. In some aspects, the methods disclosed herein improve the protein yield by at least about 250% compared to a reference method. In some aspects, the methods disclosed herein improve the protein yield by at least about 260% compared to a reference method. In some aspects, the methods disclosed herein improve the protein yield by at least about 270% compared to a reference method. In some aspects, the methods disclosed herein improve the protein yield by at least about 280% compared to a reference method. In some aspects, the methods disclosed herein improve the protein yield by at least about 290% compared to a reference method. In some aspects, the methods disclosed herein improve the protein yield by at least about 300% compared to a reference method. In some aspects, the methods disclosed herein improve the protein yield by at least about 310% compared to a reference method. In some aspects, the methods disclosed herein improve the protein yield by at least about 320% compared to a reference method. In some aspects, the methods disclosed herein improve the protein yield by at least about 330% compared to a reference method. In some aspects, the methods disclosed herein improve the protein yield by at least about 340% compared to a reference method. In some aspects, the methods disclosed herein improve the protein yield by at least about 350% compared to a reference method. In some aspects, the methods disclosed herein improve the protein yield by at least about 360% compared to a reference method. In some aspects, the methods disclosed herein improve the protein yield by at least about 370% compared to a reference method. In some aspects, the methods disclosed herein improve the protein yield by at least about 380% compared to a reference method. In some aspects, the methods disclosed herein improve the protein yield by at least about 390% compared to a reference method. In some aspects, the methods disclosed herein improve the protein yield by at least about 400% compared to a reference method.

In some aspects, the methods disclosed herein improve the protein yield by about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, or about 400% as compared to a reference method without the optimized culturing conditions disclosed herein. In some aspects, the methods disclosed herein improve the protein yield by about 100%. In some aspects, the methods disclosed herein improve the protein yield by about 110%. In some aspects, the methods disclosed herein improve the protein yield by about 120%. In some aspects, the methods disclosed herein improve the protein yield by about 130%. In some aspects, the methods disclosed herein improve the protein yield by about 140%. In some aspects, the methods disclosed herein improve the protein yield by about 150%. In some aspects, the methods disclosed herein improve the protein yield by about 160%. In some aspects, the methods disclosed herein improve the protein yield by about 170%. In some aspects, the methods disclosed herein improve the protein yield by about 180%. In some aspects, the methods disclosed herein improve the protein yield by about 190%. In some aspects, the methods disclosed herein improve the protein yield by about 200%. In some aspects, the methods disclosed herein improve the protein yield by about 210%. In some aspects, the methods disclosed herein improve the protein yield by about 220%. In some aspects, the methods disclosed herein improve the protein yield by about 230%. In some aspects, the methods disclosed herein improve the protein yield by about 240%. In some aspects, the methods disclosed herein improve the protein yield by about 250%. In some aspects, the methods disclosed herein improve the protein yield by about 260%. In some aspects, the methods disclosed herein improve the protein yield by about 270%. In some aspects, the methods disclosed herein improve the protein yield by about 280%. In some aspects, the methods disclosed herein improve the protein yield by about 290%. In some aspects, the methods disclosed herein improve the protein yield by about 300%. In some aspects, the methods disclosed herein improve the protein yield by about 310%. In some aspects, the methods disclosed herein improve the protein yield by about 320%. In some aspects, the methods disclosed herein improve the protein yield by about 330%. In some aspects, the methods disclosed herein improve the protein yield by about 340%. In some aspects, the methods disclosed herein improve the protein yield by about 350%. In some aspects, the methods disclosed herein improve the protein yield by about 360%. In some aspects, the methods disclosed herein improve the protein yield by about 370%. In some aspects, the methods disclosed herein improve the protein yield by about 380%. In some aspects, the methods disclosed herein improve the protein yield by about 390%. In some aspects, the methods disclosed herein improve the protein yield by about 400%.

In some aspects, the present methods improve the protein yield by at least about 2 fold, at least about 3 fold, at least about 4 fold, at least about 5 fold, at least about 6 fold, at least about 7 fold, at least about 8 fold, at least about 9 fold, or at least about 10 fold higher than a reference method without the optimized culturing conditions disclosed herein. In some aspects, the present methods improve the protein yield by at least about 2 fold. In some aspects, the present methods improve the protein yield by at least about 3 fold. In some aspects, the present methods improve the protein yield by at least about 4 fold. In some aspects, the present methods improve the protein yield by at least about 5 fold. In some aspects, the present methods improve the protein yield by at least about 6 fold. In some aspects, the present methods improve the protein yield by at least about 7 fold. In some aspects, the present methods improve the protein yield by at least about 8 fold. In some aspects, the present methods improve the protein yield by at least about 9 fold. In some aspects, the present methods improve the protein yield by at least about 10 fold

In some aspects, the present methods improve the protein yield by about 2 fold, about 3 fold, about 4 fold, about 5 fold, about 6 fold, about 7 fold, about 8 fold, about 9 fold, or about 10 fold higher than a reference method without the optimized culturing conditions disclosed herein. In some aspects, the present methods improve the protein yield by about 2 fold. In some aspects, the present methods improve the protein yield by about 3 fold. In some aspects, the present methods improve the protein yield by about 4 fold. In some aspects, the present methods improve the protein yield by about 5 fold. In some aspects, the present methods improve the protein yield by about 6 fold. In some aspects, the present methods improve the protein yield by about 7 fold. In some aspects, the present methods improve the protein yield by about 8 fold. In some aspects, the present methods improve the protein yield by about 9 fold. In some aspects, the present methods improve the protein yield by about 10 fold. In some aspects, the present methods improve the protein yield by about 2 fold to about 3 fold. In some aspects, the present methods improve the protein yield by about 3 fold to about 4 fold. In some aspects, the present methods improve the protein yield by about 4 fold to about 5 fold. In some aspects, the present methods improve the protein yield by about 5 fold to about 6 fold. In some aspects, the present methods improve the protein yield by about 6 fold to about 7 fold. In some aspects, the present methods improve the protein yield by about 7 fold to about 8 fold. In some aspects, the present methods improve the protein yield by about 8 fold to about 9 fold.

In some aspects, the values of product quality attributes of the recombinant protein obtained by transiently transfecting the eukaryotic cells is within ±10% of the values of product quality attributes of the recombinant protein obtained by stable transfection. In some aspects, the values of product quality attributes of the recombinant protein obtained by transiently transfecting the eukaryotic cells is within ±15% of the values of product quality attributes of the recombinant protein obtained by stable transfection. In some aspects, the values of product quality attributes of the recombinant protein obtained by transiently transfecting the eukaryotic cells is within ±20% of the values of product quality attributes of the recombinant protein obtained by stable transfection.

In some aspects, the product quality attributes are selected from the group consisting of
(i) protein aggregation;
(ii) reduced and non-reduced species;
(iii) charge variants;
(iv) glycosylation profile; and,
(v) any combination thereof.

In some aspects, the protein aggregation quality attributes are selected from the group consisting of
(i) percentage of high molecular weight species (HMW %),
(ii) percentage of monomeric species, and
(iii) any combination thereof.

In some aspects, the protein aggregation quality attributes are determined using high performance liquid size exclusion chromatography (HPLC-SEC). For example, the HMW and monomeric species levels can be measured by HPLC-SEC using an Alliance 2695 HPLC system equipped with a Model 2487 dual wavelength detector (Waters Corporation, Milford MA USA) and TSKgel SuperSW3000 main and guard columns (Tosoh Bioscience, King of Prussia PA USA). As used herein, “high molecular weight species” or “HMW” refer to aggregated of the recombinant proteins comprising two or more monomeric species. For example, a HMW species can be a dimer, a trimer, a tetramer, a pentamer, or a hexamer.

In some aspects, percentage of high molecular weight species (HMW %), percentage of monomeric species, or any combination thereof of recombinant proteins produced using the transient expression methods disclosed have the same value ±5%, ±10%, or ±20%, with respect to the values observed when the recombinant protein is produced using a stable transfection reference method.

In some aspects, the reduced and non-reduced species quality attributes are selected from the group consisting of
(i) percentage of reduced recombinant protein,
(ii) percentage of non-reduced recombinant protein, and
(iii) any combination thereof.

In some aspects, the reduced and non-reduced species quality attributes are determined using capillary electrophoresis under reducing and non-reducing conditions (CE-SDS). In some aspects, percentage of reduced recombinant protein, percentage of non-reduced recombinant protein, or any combination thereof of recombinant proteins produced using the transient expression methods disclosed have the same value ±5%, ±10%, or ±20%, with respect to the values observed when the recombinant protein is produced using a stable transfection reference method.

In some aspects, the charge variants quality attributes are selected from the group consisting of
(i) percentage of basic variants,
(ii) percentage of acidic variants,
(iii) percentage of main species, and
(iv) any combination thereof.

As used herein, the term “basic variants” refers to forms of the recombinant protein that have more positive charges than the main species. As used herein, the term “acidic variants” refers to forms of the recombinant protein that have more negative charges than the main species. In some aspects, the charge variants quality attributes are determined by analyzing isoelectric distribution isoelectric focusing, e.g., imaged capillary isoelectric focusing (iCIEF). iCIEF refers to a method used for the separation of proteins by their isoelectric point (pI). In this method, samples are prepared to a final concentration of approximately 1 mg/mL with water, methyl cellulose, ampholytes, and pI markers, and then injected by an autosampler into an imaged capillary isoelectric focusing system. Electrophoresis separates the samples through a pH gradient within a fluorocarbon (FC) coated capillary based on charge variance of the isoforms/variants. Results are then compared to reference separations to compare and characterize groups of components.

In some aspects, the percentage of basic variants, percentage of acidic variants, percentage of main species, or any combination thereof quality attributes are determined using isoelectric focusing, e.g., imaged capillary isoelectric focusing (iCIEF). In some aspects, percentage of basic variants, percentage of acidic variants, percentage of main species, or any combination thereof of recombinant proteins produced using the transient expression methods disclosed have the same value ±5%, ±10%, or ±20%, with respect to the values observed when the recombinant protein is produced using a stable transfection reference method.

As used herein, “glycosylation profile” refers the amounts of N-linked or O-linked sugar residues covalently attached to a protein molecule, such as a glycoprotein.

As used herein, the term “glycoprotein” refers to a protein that is modified by the addition of one or more carbohydrates, including the addition of one or more sugar residues. As used herein, “glycosylation” refers to the addition of complex oligosaccharide structures to a protein at specific sites within the polypeptide chain. Glycosylation of proteins and the subsequent processing of the added carbohydrates can affect protein folding and structure, protein stability, including protein half-life, and functional properties of a protein. Protein glycosylation can be divided into two classes by virtue of the sequence context where the modification occurs, O-linked glycosylation and N-linked glycosylation. O-linked polysaccharides are linked to a hydroxyl group, usually to the hydroxyl group of either a serine or a threonine residue. O-glycans are not added to every serine and threonine residue. O-linked oligosaccharides are usually mono or biantennary, i.e. they comprise one or at most two branches (antennas), and comprise from one to four different kinds of sugar residues, which are added one by one. N-linked polysaccharides are attached to the amide nitrogen of an asparagine. Only asparagines that are part of one of two tripeptide sequences, either asparagine-X-serine or asparagine-X-threonine (where X is any amino acid except proline), are targets for glycosylation. N-linked oligosaccharides can have from one to four branches referred to as mono-, bi-, tri-tetraantennary. The structures of and sugar residues found in N- and O-linked oligosaccharides are different. Despite that difference, the terminal residue on each branch of both N- and O-linked polysaccharide can be modified by a sialic acid molecule a modification referred as sialic acids capping. Sialic acid is a common name for a family of unique nine-carbon monosaccharides, which can be linked to other oligosaccharides. Two family members are N-acetyl neuraminic acid, abbreviated as Neu5Ac, NeuAc, or NANA, and N-glycolyl neuraminic acid, abbreviated as Neu5Gc or NGNA. The most common form of sialic acid in humans is NANA. N-acetylneuraminic acid (NANA) is the primary sialic acid species present in CTLA4-Ig molecules. However, it should be noted that minor but detectable levels of N glycolylneuraminic acid (NGNA) are also present in CTLA4-Ig molecules. Furthermore, the method described herein can be used to determine the number of moles of sialic acids for both NANA and NGNA, and therefore levels of both NANA and NGNA are determined and reported for CTLA4-Ig molecules. N- and O-linked oligosaccharides have different number of branches, which provide different number of positions to which sialic acid molecules can be attached. N-linked oligosaccharides can provide up to four attachment positions for sialic acids, while O-linked oligosaccharides can provide two sites for sialic acid attachment.

In some aspects, the glycosylation profile comprises one or more N-linked glycans. As used herein “N-linked glycan” refers to a protein modification where a glycan is linked to a glycoconjugate via a nitrogen linkage. The acceptors of the glycan are selected asparagine residues of polypeptide chains that have entered the periplasm or the lumen of the ER, respectively. Oligosaccharyltransferase, the central enzyme of the N-glycosylation pathway, catalyzes the formation of an N-glycosidic linkage of the oligosaccharide to the side-chain amide of asparagine residues that are specified by the consensus sequence N-X-S/T. All eukaryotic N-glycans share a common core sequence, Manα1-3(Manα1-6)Manβ1-4GlcNAcβ1-4GlcNAcβ1-Asn-X-Ser/Thr, and are classified into three types: (1) oligomannose, in which only Man residues extend the core; (2) complex, in which “antennae” initiated by GlcNAc extend the core; and (3) hybrid, in which Man extends the Manα1-6 arm of the core and one or two GlcNAcs extend the Manα1-3 arm.

As used herein, “N-linked glycosylation” refers the attachment of oligosaccharides to a nitrogen atom, usually the N4 of asparagine residues. N-glycosylation can occur on secreted or membrane bound proteins, mainly in eukaryotes and archaea. A detailed review of the biosynthetic pathways and enzymes used to generate N-linked glycans (e.g., high mannose type oligosaccharides) are described in Stanley et al., “N-Glycans” in Essentials of Glycobiology, Ed. Varki, Cummings, and Eskho, Cold Spring Harbor Press, 2009.

In some aspects, the N-linked glycans comprise (mannose-3-N-acetylglucosamine-4-fucose) (G0F), mannose-3-N-acetylglucosamine-4-galactose-1-fucose (G1F), mannose-3-N-acetylglucosamine-4-galactose-2-fucose (G2F), mono-sialylated mannose-3-N-acetylglucosamine-4-galactose-1-fucose (S1G1F), mono-sialylated mannose-3-N-acetylglucosamine-4-galactose-2-fucose (S1G2F), mono-sialylated mannose-3-N-acetylglucosamine-4-galactose-3-fucose (S2G3F), di-sialylated mannose-3-N-acetylglucosamine-4-galactose-2-fucose (S2G2F), or any combination thereof. In some aspects, the glycosylation profile quality attributes are selected from the group consisting (i) percentage of G1F, (ii) percentage of G0F, (iii) percentage of G2F, (iv) percentage of total afucosylated protein, and (v) any combination thereof. In some aspects, the glycosylation profile quality attributes are determined using HPLC methods, for example, Ultra Performance Liquid Chromatography with fluorescence detection (UPLC-FLR).

In some aspects, (i) percentage of G1F, (ii) percentage of G0F, (iii) percentage of G2F, (iv) percentage of total afucosylated protein, and (v) any combination thereof of recombinant proteins produced using the transient expression methods disclosed have the same value ±5%, ±10%, or ±20%, with respect to the values observed when the recombinant protein is produced using a stable transfection reference method.

The present disclosure provides also a recombinant protein obtained according to the methods disclosed herein. In some aspects, the recombination protein is a fusion protein consisting, e.g., of an immunoglobulin component (e.g. the Fc component) and a growth factor (e.g. an interleukin), an antibody or any antibody derived molecule formats or an antibody fragment, and antigen-binding fragment. In some aspects, the recombinant protein comprises a naturally occurring protein. In other aspects, the recombinant protein can be an antibody, an enzyme, a cytokine, a lymphokine, an adhesion molecule, a receptor, or any derivative or fragments thereof. In some aspects, the recombination protein is an agonists or an antagonists and/or have therapeutic or diagnostic use or can be used as a research reagent. In some aspects, the polypeptide is a secreted protein or protein fragment, e.g., an antibody or antibody fragment or an Fc-fusion protein.

In some specific aspects, the recombinant protein comprises an antibody or an antigen-binding portion thereof, e.g., a monoclonal antibody (mAb). In some aspects, the antibody or antigen-binding fragment binds an antigen selected from the group consisting of PD-1, PD-L1, CVTLA-4, LAG-3, TIGIT, GITR, CXCR4, CD73, HER2, VEGF, CD20, CD40, CD11a, tissue factor (TF), PSCA, IL-8, EGFR, HER3, and HER4.

The present disclosure also provide pharmaceutically compositions comprising a recombinant protein or combination thereof obtained according to the methods disclosed herein and a pharmaceutically acceptable carrier. As used herein, “pharmaceutically acceptable carrier” refers to a vehicle for a pharmacologically active agent, e.g., a recombinant protein produced according to the methods disclosed herein. The carrier facilitates delivery of the active agent to the target site without terminating the function of the agent. Non-limiting examples of suitable forms of the carrier include solutions, creams, gels, gel emulsions, jellies, pastes, lotions, salves, sprays, ointments, powders, solid admixtures, aerosols, emulsions (e.g., water in oil or oil in water), gel aqueous solutions, aqueous solutions, suspensions, liniments, tinctures, and patches suitable for topical administration.

Also provided, is a cell or plurality of cells obtained according to the methods disclosed. The cells used in the methods disclosed herein are eukaryotic cells. In some aspects, the eukaryotic cells are mammalian cells, e.g., mammalian cells selected from the group consisting of Chinese Hamster Ovary (CHO), VERO, BHK, HEK, HeLa, COS, MDCK, and hybridoma cells. In some specific aspects, the mammalian cells are CHO cells.

Mammalian cells that be used according to the methods disclosed herein include, e.g., DHFR-CHO cells, described in Urlaub and Chasin, (1980) PNAS USA 77:4216-4220, NSO myeloma cells, COS cells and SP2 cells, monkey kidney CV1 line transformed by SV40 (COS-7, ATCC CRL 1651); human embryonic kidney (HEK) lines (293 or 293 cells subcloned for growth in suspension culture); baby hamster kidney cells (BHK, ATCC CCL 10); mouse sertoli cells (TM4); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; human hepatoma line cells (Hep G2), NIH 3T3, W138, BT483, Hs578T, HTB2, BT2O, T47D, CRL7O3O, COS (e.g., COS1 or COS), PER.C6, HsS78Bst, HEK-293T, SP210, R1.1, B-W, L-M, BSC1, BSC40, YB/20, BMT10, and HsS78Bst cells.

In some aspects, the CHO cell is CHO-DG44, CHOZN, CHO/DHFR-, CHOK1SV GS-KO, or CHO-S. In some aspects, the CHO cell is CHO-DG4. In some aspects, the CHO cell is CHOZN. Other suitable CHO cell lines disclosed herein include CHO-K (e.g., CHO K1), CHO pro3-, CHO P12, CHO-K1/SF, DUXB11, CHO DUKX; PA-DUKX; CHO pro5; DUK-BII or derivatives thereof.

In some aspects, the eukaryotic cells are eukaryotic microbes such as filamentous fungi or yeast. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; Yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

In some aspects, the eukaryotic cells are derived from multicellular organisms. In some aspects, the cells are invertebrate cells from plant and insect cells. Non-limiting examples include cells derived from Spodoptera frugiperda (caterpillar), Aedes aegypti (mosquito), Aedes albopictus (mosquito), Drosophila melanogaster (fruitfly), Bombyx mori, cotton, corn, potato, soybean, petunia, tomato, and tobacco can also be utilized.

The present disclosure also provides a bioreactor for the manufacture of a recombinant protein produced according to the methods disclosed herein. Also provided is a bioreactor comprising a cell or plurality of cells transfected with cells as described herein. Also provided is a bioreactor comprising transfected cells or plurality of transfected cells disclosed herein, cultured under the conditions described herein. Specific types of bioreactor that can be used according to the methods disclosed herein, e.g., a high-throughput bioreactor such as an AMBR® 250 bioreactor, or a large scale (e.g., 5L) bioreactor are described in detail above.

In some aspects, the methods of the present disclosure can be used to produce a recombinant protein. In some aspects, the methods of the present disclosure can be used for producing AAV vectors useful for gene therapy. In some aspects, the methods can be used for AAV vector manufacturing lots and small batches for personalized biologic therapeutics.

### II. Recombinant Proteins

The methods disclosed herein can be used for the production of recombinant proteins, e.g., therapeutic proteins. In some aspects, the proteins produced by the methods according to the present disclosure are antibodies. Antibodies can include, for example, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), camelized antibodies, affybodies, Fab fragments, F(ab′)2 fragments, disulfide-linked Fvs (sdFv), anti-idiotypic (anti-Id) antibodies (including, e.g., anti-anti-Id antibodies), and antigen-binding fragments of any of the above. In certain aspects, antibodies described herein refer to polyclonal antibody populations. Antibodies can be of any type (e.g., IgG, IgE, IgM, IgD, IgA or IgY), any class (e.g., IgG1, IgG2, IgG3, IgG4, IgA1 or IgA2), or any subclass (e.g., IgG2a or IgG2b) of immunoglobulin molecule. In certain aspects, antibodies described herein are IgG antibodies, or a class (e.g., human IgG1 or IgG4) or subclass thereof. In a specific aspect, the antibody is a humanized monoclonal antibody. In another specific aspect, the antibody is a human monoclonal antibody, preferably that is an immunoglobulin. In certain aspects, an antibody described herein is an IgG1, or IgG4 antibody.

In some aspects, the protein described herein is an “antigen-binding domain,” “antigen-binding region,” “antigen-binding fragment,” and similar terms, which refer to a portion of an antibody molecule which comprises the amino acid residues that confer on the antibody molecule its specificity for the antigen (e.g., the complementarity determining regions (CDR)). The antigen-binding region can be derived from any animal species, such as rodents (e.g., mouse, rat or hamster) and humans.

In some aspects, the protein is an anti-LAG3 antibody, an anti-CTLA-4 antibody, an anti-TIM3 antibody, an anti-NKG2a antibody, an anti-ICOS antibody, an anti-CD137 antibody, an anti-KIR antibody, an anti-TGFβ antibody, an anti-IL-10 antibody, an anti □B7-H4 antibody, an anti-Fas ligand antibody, an anti-mesothelin antibody, an anti-CD27 antibody, an anti-GITR antibody, an anti-CXCR4 antibody, an anti-CD73 antibody, an anti-TIGIT antibody, an anti-OX40 antibody, an anti-PD-1 antibody, an anti-PD-L1 antibody, an anti-IL8 antibody, or any combination thereof. In some aspects, the protein is Abatacept NGP. In other aspects, the protein is Belatacept NGP. In some aspects, the protein is an anti-GITR (glucocorticoid-induced tumor necrosis factor receptor family-related gene) antibody. In some aspects, the anti-GITR antibody has the CDR sequences of 6C8, e.g., a humanized antibody having the CDRs of 6C8, as described, e.g., in WO2006/105021; an antibody comprising the CDRs of an anti-GITR antibody described in WO2011/028683; an antibody comprising the CDRs of an anti-GITR antibody described in JP2008278814, an antibody comprising the CDRs of an anti-GITR antibody described in WO2015/031667, WO2015/187835, WO2015/184099, WO2016/054638, WO2016/057841, WO2016/057846, WO 2018/013818, or other anti-GITR antibody described or referred to herein, all of which are incorporated herein in their entireties. In other aspects, the protein is an anti-LAG3 antibody. Lymphocyte-activation gene 3, also known as LAG-3, is a protein which in humans is encoded by the LAG3 gene. LAG3, which was discovered in 1990 and is a cell surface molecule with diverse biologic effects on T cell function. It is an immune checkpoint receptor and as such is the target of various drug development programs by pharmaceutical companies seeking to develop new treatments for cancer and autoimmune disorders. In soluble form it is also being developed as a cancer drug in its own right. Examples of anti-LAG3 antibodies include, but are not limited to, the antibodies in WO 2017/087901 A2, WO 2016/028672 A1, WO 2017/106129 A1, WO 2017/198741 A1, US 2017/0097333 A1, US 2017/0290914 A1, and US 2017/0267759 A1, all of which are incorporated herein in their entireties. In some aspects, the protein is an anti-CXCR4 antibody. CXCR4 is a 7 transmembrane protein, coupled to G1. CXCR4 is widely expressed on cells of hemopoietic origin, and is a major co-receptor with CD4+ for human immunodeficiency virus 1 (HIV-1) See Feng, Y., Broeder, C. C., Kennedy, P. E., and Berger, E. A. (1996) Science 272, 872-877. Examples of anti-CXCR4 antibodies include, but are not limited to, the antibodies in WO 2009/140124 A1, US 2014/0286936 A1, WO 2010/125162 A1, WO 2012/047339 A2, WO 2013/013025 A2, WO 2015/069874 A1, WO 2008/142303 A2, WO 2011/121040 A1, WO 2011/154580 A1, WO 2013/071068 A2, and WO 2012/175576 A1, all of which are incorporated herein in their entireties. In some aspects, the protein is an anti-CD73 (ecto-5′-nucleotidase) antibody. In some aspects, the anti-CD73 antibody inhibits the formation of adenosine. Degradation of AMP into adenosine results in the generation of an immunosuppressed and pro-angiogenic niche within the tumor microenvironment that promotes the onset and progression of cancer. Examples of anti-CD73 antibodies include, but are not limited to, the antibodies in WO 2017/100670 A1, WO 2018/013611 A1, WO 2017/152085 A1, and WO 2016/075176 A1, all of which are incorporated herein in their entireties. In some aspects, the protein is an anti-TIGIT (T cell Immunoreceptor with Ig and ITIM domains) antibody. TIGIT is a member of the PVR (poliovirus receptor) family of immunoglobin proteins. TIGIT is expressed on several classes of T cells including follicular B helper T cells (TFH). The protein has been shown to bind PVR with high affinity; this binding is thought to assist interactions between TFH and dendritic cells to regulate T cell dependent B cell responses. Examples of anti-TIGIT antibodies include, but are not limited to, the antibodies in WO 2016/028656 A1, WO 2017/030823 A2, WO 2017/053748 A2, WO 2018/033798 A1, WO 2017/059095 A1, and WO 2016/011264 A1, all of which are incorporated herein by their entireties. In some aspects, the protein is an anti-OX40 (i.e., CD134) antibody. OX40 is a cytokine of the tumor necrosis factor (TNF) ligand family. OX40 functions in T cell antigen-presenting cell (APC) interactions and mediates adhesion of activated T cells to endothelial cells. Examples of anti-OX40 antibodies include, but are not limited to, WO 2018/031490 A2, WO 2015/153513 A1, WO 2017/021912 A1, WO 2017/050729 A1, WO 2017/096182 A1, WO 2017/134292 A1, WO 2013/038191 A2, WO 2017/096281 A1, WO 2013/028231 A1, WO 2016/057667 A1, WO 2014/148895 A1, WO 2016/200836 A1, WO 2016/100929 A1, WO 2015/153514 A1, WO 2016/002820 A1, and WO 2016/200835 A1, all of which are incorporated herein by their entireties. In some aspects, the protein is an anti-IL8 antibody. IL-8 is a chemotactic factor that attracts neutrophils, basophils, and T-cells, but not monocytes. It is also involved in neutrophil activation. It is released from several cell types in response to an inflammatory stimulus.

In some aspects, the proteins produced by the methods according to the present disclosure are fusion proteins. A “fusion” or “fusion” protein comprises a first amino acid sequence linked in frame to a second amino acid sequence with which it is not naturally linked in nature. The amino acid sequences which normally exist in separate proteins can be brought together in the fusion polypeptide, or the amino acid sequences which normally exist in the same protein can be placed in a new arrangement in the fusion polypeptide. A fusion protein is created, for example, by creating and translating a polynucleotide in which the peptide regions are encoded in the desired relationship. Upon transcription/translation, a single protein is made. In this way, multiple proteins, or fragments thereof can be incorporated into a single polypeptide.

In some aspects, the protein is Abatacept (marketed as ORENCIA®). Abatacept (also abbreviated herein as Aba) is a drug used to treat autoimmune diseases like rheumatoid arthritis, by interfering with the immune activity of T cells. Abatacept is a fusion protein composed of the Fc region of the immunoglobulin IgG1 fused to the extracellular domain of CTLA-4. In order for a T cell to be activated and produce an immune response, an antigen presenting cell must present two signals to the T cell. One of those signals is the major histocompatibility complex (MHC), combined with the antigen, and the other signal is the CD80 or CD86 molecule (also known as B7-1 and B7-2).

In some aspects, the protein is Belatacept (trade name NULOJIX®). Belatacept is a fusion protein composed of the Fc fragment of a human IgG1 immunoglobulin linked to the extracellular domain of CTLA-4, which is a molecule crucial in the regulation of T cell costimulation, selectively blocking the process of T-cell activation. It is intended to provide extended graft and transplant survival while limiting the toxicity generated by standard immune suppressing regimens, such as calcineurin inhibitors. It differs from abatacept (ORENCIA®) by only 2 amino acids.

### III. System

The present disclosure provides a system for high-yield production of a recombinant protein by transient transfection comprising:
(i) a bioreactor;
(ii) an ATF perfusion system; and,
(iii) an electroporation transfection system.

In some aspects, the bioreactor is a high-throughput bioreactor such as an AMBR® 250 bioreactor. In some aspects, the bioreactor is a large-volume bioreactor such as a 5L bioreactor.

In some aspects, the bioreactor, e.g., a high-throughput bioreactor such as an AMBR®250 bioreactor comprises at least one integrated cell culture analyzer. In some aspects, the cell culture analyzer is a pH measurement module, e.g., an AMBR® Analysis module. In some aspects, the cell culture analyzer is an integrated cell counter, e.g., a Beckman Vicell XR cell counter or a Cedex HiRes cell counter. In some aspects, the cell culture analyzer comprises a Nova Biomedical FLEX2 External Sampling Module (ESM), and a FLEX2 Analyzer, or a combination thereof. In some aspects, automated sample, sample transfer and analysis, data transfer and automated feedback control are conducted by a cell culture analyzer, e.g., a FLEX2 Analyzer. In some aspects, control actions can be executed by the cell culture analyzer, e.g., a FLEX2 Analyzer such as glucose or feed addition base on glucose level, cell count, viable cell count, or other parameters.

In some aspects, the ATF perfusion system is a REPLIGEN™ ATF system. In some aspects, the REPLIGEN™ ATF perfusion system is an Xcell ATF® 2 system. In some aspects, the REPLIGEN™ ATF perfusion system is an Xcell ATF® 1 system. In some aspects, the REPLIGEN™ ATF perfusion system is an Xcell ATF® 4 system (10 L to 50 L suspension culture volume). In some aspects, the REPLIGEN™ ATF perfusion system is an Xcell ATF® 10 system (50 L to 200 L suspension culture volume).

In some aspects, the REPLIGEN™ ATF perfusion system is connected to a REPLIGEN™ XCell™ Lab Controller. In some aspects, the REPLIGEN™ ATF perfusion system is connected to a REPLIGEN™ XCell™ C410 Controller.

In some aspects, the electroporation transfection system is a flow-based electroporation system. In some aspects, the flow-based electroporation system is a MAXCYTE™ STX® flow transfection system. In some aspects, the flow-based electroporation system is a MAXCYTE™ VLS® flow transfection system. In some aspects, the flow-based electroporation system is a MAXCYTE™ GT® flow transfection system.

### IV. Pharmaceutical Compositions

The proteins produced by the methods of the present disclosure can be further formulated to be suitable for human administration, e.g., pharmaceutical composition. A composition that is acceptable for pharmaceutical administration, such a composition disclosed herein can include substances that are impurities at a level not exceeding an acceptable level for pharmaceutical administration (such level including an absence of such impurities), and can include pharmaceutically acceptable excipients, vehicles, carriers and other inactive ingredients, for example, to formulate such composition for ease of administration, in addition to any active agent(s).

Various aspects of the disclosure are described in further detail in the following subsections. The present disclosure is further illustrated by the following examples which should not be construed as further limiting.

### Examples

### Materials and Methods

### I. Cell Culture

Proprietary suspension-adapted BMS-CHO1 cells were cultured in proprietary expansion media with 8-12 mM L-Glutamine (Ajinomoto) in non-baffled shake flasks and incubated at 37° C. under 5% CO₂ and 150 rpm. Cells were passaged every 2-3 days at a seeding density of 3-4×10⁵ cells/ml for up to 7-11 passages.

For initial small scale shake flask studies for screening different conditions, cells were split in a 1:3 ratio at 3-4×10⁶ cells/ml one day prior to electroporation. Post the initial optimization phase, an alternating tangential flow filtration (ATF)-based perfusion system was optimized to achieve high cell densities needed for flow-based electroporation. For this, cells were inoculated in 5 L Applikon Biotechnology (Sartorius) stirred tank bioreactors at 3-3.5×10⁵ cells/ml, controlled by Finesse G3LAB™ Universal TRUEBIO™ (Sartorius) control systems and software. 24 hours post inoculation, cells were perfused at 0.8 cell specific perfusion rate (CSPR) using the XCELL™ ATF2™ system (Repligen) for the remaining 3 days of culture. At the conclusion of the perfusion phase, cell densities reached 40-60×10⁶ cells/ml. To reach the densities of 200×10⁶ cells/ml required for electroporation, the medium replenishment pump was stopped, and permeate pumping was continued for 30-45 min, in which time the cell culture concentration reached 80-100×10⁶ cells/ml. Cells were then centrifuged as described in the following section.

### II. Transfection

The day of transfection, cells are pelleted by centrifugation at 1,000 rpm for 5-10 min and suspended at 200×10⁶ cells/ml in electroporation buffer (MaxCyte, Gaithersburg, MD). Cell suspensions were mixed with plasmid DNA (1.5 μg DNA per 1×10⁶ cells) and then transferred to CL-1.1 (small scale electroporation) or CL-2 (large scale electroporation) processing assemblies (MaxCyte, Gaithersburg, MD). The loaded processing assembly was plugged onto the MaxCyte STX Transfection System previously set with the “CHO” protocol provided with the instrument. Immediately after electroporation, cells were transferred to a shake flask and incubated for 30-40 min at 37° C. in a 5% CO₂ in a static incubator.

### III. Production Run in Shake Flasks

Cells were resuspended in a proprietary media with 8-12 mM L-Glutamine in non-baffled shake flasks and incubated at 37 C in a 5% CO₂ incubator at 90 rpm (25 mm shaking diameter). 24 hours post-electroporation, shaking speed was increased to 110 rpm. Cells were fed with proprietary feed A everyday up to day 4, and then every other day until harvest. Glucose (300 g/L, VWR) was supplemented to 4 g/L when glucose level was below 3 g/L and anti-clumping (ThermoFisher) was added at a 1:200 ratio on day 3 or 4. During the optimization process, various initial cell concentrations, media supplements, and temperature shift strategies were tested as described in the Results and Discussion section.

### IV. Production Run in AMBR® 250 Reactor

For inoculation of the transient gene expression (TGE) screening experiments an AMBR® 250 (Sartorius, New York) automated bioreactor system was used. The AMBR® 250 system is a high throughput, automated bioreactor system for process development with 12 or 24 fully featured single-use 100-250 mL mini bioreactors.

Cells were inoculated in 24 separate single-use 250 ml bioreactors according to conditions outlined in the experimental plan. Feeding and sampling were automated, as were cell counts and metabolite data collection by the addition of Vi-Cell (Beckman, California) and Nova Bioprofile Flex2 (Nova Biomedical, Massachusetts) instruments to the AMBR® 250 system. Daily supplemental feeding was controlled via pump addition. Other initial additions such as N, N-Dimethyl acetamide (DMA, Millipore Sigma) and Sodium Butyrate (NaBu, Millipore Sigma) were manually pipetted. Culture duration was 14 days, and samples were collected regularly for offline protein quality and titer analysis.

### V. Production Run in 5-L Glass Tank Bioreactors

For inoculation of the scale-up experiment to test optimal conditions from the TGE screen, 5 L Applikon (Sartorius) stirred tank bioreactors were utilized in a similar manner to those used previously in preparation for intensification via ATF. Cells were inoculated and controlled according to the parameters set in TABLE 2. pH and DO control were achieved through amperometric and optical probes (Mettler Toledo) and tied to control loops to supplement oxygen, carbon dioxide, and sodium carbonate (Jost Chemical). Feeding was automatically controlled via the multifeed control system (Sartorius). Supplemental feed media, glucose, and antifoam-C were all fed in this manner: (i) feed media was provided as a fixed percentage daily, (ii) glucose was added to 3 g/L pre-feed, and (iii) antifoam-C was added to 3 ppm daily and as needed for foam control, not to exceed 100 ppm total. Daily samples were manually taken from the vessels for product quality retains and to determine cell counts and metabolite data.

### VI. Cell Counts and Metabolites

Viable Cell Density (VCD) and viability were measured off-line using a VI-CELL® XR automatic cell counter (Beckman Coulter). Whole broth cell-culture samples were used for off-line gas assays (pH, pCO2 and pO2) and for off-line metabolite assays (glucose, glutamine, glutamate, ammonium, and lactate) using a BIOPROFILE® FLEX2™ multitest cell culture analyzer (Nova Biomedical). Clarified supernatant after centrifugation at 1,000 rpm was used for LDH quantification using a CEDEX® BIO HT® bioprocess analyzer (Roche).

### VII. Cell Cycle and Apoptosis Assessment

Cell cycle analysis was performed using the GUAVA® Cell Cycle Reagent (Luminex) following the manufacturer's instructions. Briefly, cells were diluted to 2×10⁵ cells/sample in 1×PBS buffer, centrifuged at 500×g for 5 min and then washed once with 1×PBS. Cells were resuspended in residual buffer, and 70% cold ethanol (Thermo Fisher) was added slowly. Cells were incubated at −20 C at least overnight before washing them with 1×PBS. Cells were stained with GUAVA® Cell Cycle Reagent (Luminex) containing propidium iodide (PI) and incubated for 30 min at room temperature, light protected. Stained cells were in analyzed in a GUAVA® EASYCYTE™ flow cytometer (Luminex)

Early and late apoptosis were assayed with cells diluted to 1×10⁵ cells/sample in 1×PBS buffer. FBS (Thermo Fisher) was added to 10% (v/v) and then stained for 20 min with GUAVA NEXIN® Reagent Kit (Luminex) following the manufacturer's instructions. After incubation, cells are analyzed in a GUAVA® EASYCYTE™ flow cytometer (Luminex)

### VIII. Protein Quality Analysis

After conclusion of the production phase, culture was centrifuged at 1,000×g (Beckman Allegra X-12) for 10 minutes. Culture was then diluted to less than 2 mg/ml and loaded for plate-based purification in MABSELECT™ SURE™ PCC resin (Cytiva Life Sciences). The plate was then washed 3× and eluted in acetate buffer, neutralized in 2M tris, and read at A280 nm for protein concentration. Titer was measured via protein A UPLC method, and the purified sample was characterized for purity using high performance liquid chromatography-size exclusion chromatography (HPLC-SEC) and reduced/non-reduced capillary electrophoresis (CE-SDS) using Caliper LC90 CE-SDS gel technology. Protein quality was assayed by analyzing isoelectric distribution by imaged capillary isoelectric focusing (icIEF), and quantification of N-linked glycan species by HPLC methods.

### Results and Discussion

### TGE Process Screening Resulted in Multi-Fold Improvement in Transient Gene Expression.

Several cell culture process parameters were screened, including inoculation seeding density, addition of Sodium Butyrate (NaBu), supplementation of N, N-Dimethyl acetamide (DMA), temperature shift timing and amount of DNA transfected. A Box-Behnken design was generated with the R package ‘rsm’ using four factors (seeding density, NaBu, DMA and temp. shift timing) with three levels each, resulting in 26 different conditions. Fed-batch runs were performed in 250-mL shake flasks with 60 mL working volume. The performance of transient gene expression was assessed based on normalized titer, expressed as the titer relative to the baseline condition (Seeding density: 5×10⁶ cells/mL, NaBu: 1 mM, DMA: 0 mM, and Temp. shift time: 24 h).

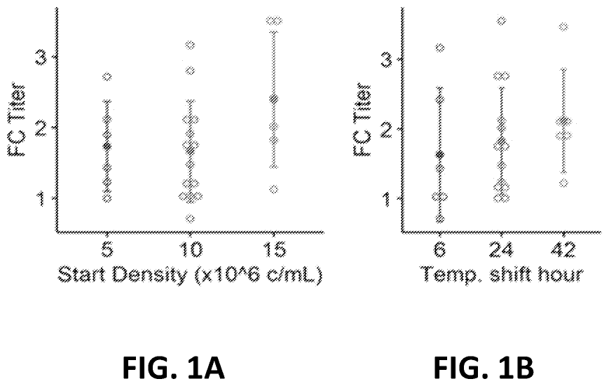

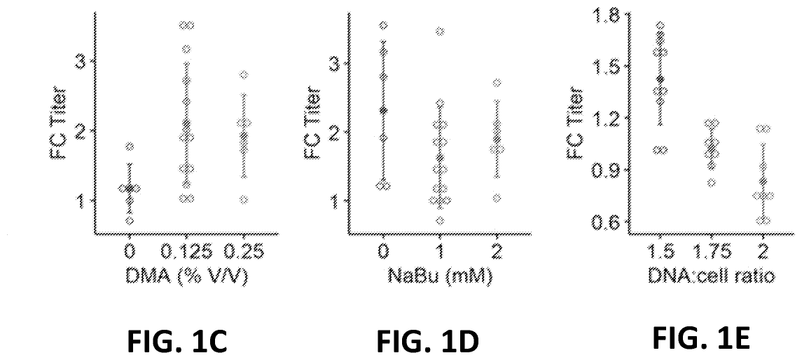

The addition of DMA was found to be statistically significant (p-value <0.01) and the DMA concentration of 0.125% V/V produced the highest averaged normalized titer (FIG. 1A). The process development strategy led to a multi-fold increase in final titer as seen in FIGS. 1A-1E. By analyzing the individual effects, it was found that higher seeding densities favored higher titers (FIG. 1B). Similarly, within the times tested, the titer increased with later temperature shift times (FIG. 1C). The addition of NaBu was detrimental to the titer in the fed-batch shake flasks (FIG. 1D). We additionally screened the effect of increasing amounts of DNA (expressed as μg of DNA per 10⁶ cells/mL) and found that higher DNA to cell ratios (i.e., DNA: cell ratio of 1.75 and 2 as μg of DNA per 10⁶ cells/mL) had a substantial undesirable impact on final titer (FIG. 1E).

### Continuous ATF Perfusion for Cell Intensification and Concentration Improved Cell Culture Attributes and Titer

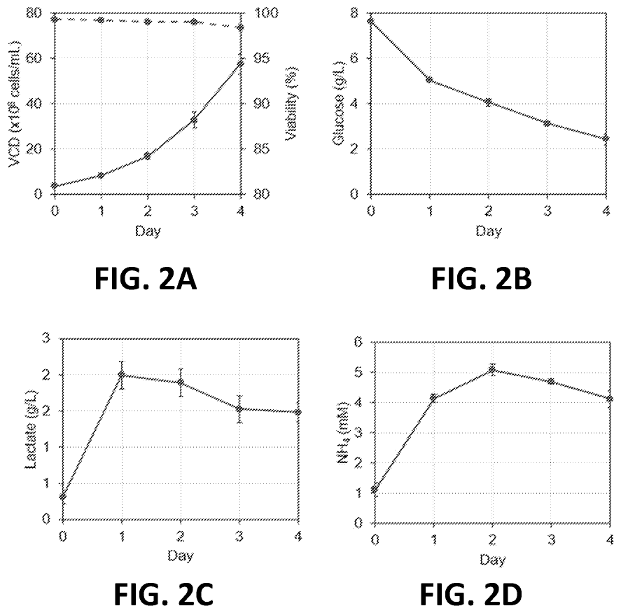

Given that the screening study above indicated that high seeding densities favor higher titer, an N−1 perfusion was introduced into the seed process to leverage large numbers of host cells to facilitate the development of an intensified fed-batch process platform for TGE. The N−1 seed culture was run in a perfusion mode using a proprietary enriched media for four days. The bioreactor performance for N−1 seed perfusion and the in-process attributes are shown in FIGS. 2A-2D.

For N−1 perfusion cultures, the bioreactor achieved final viable cell density (VCD) of approximately 60×10⁶ cells/ml with viability higher than 98% for the entire 4-day duration (FIG. 2A). Measured metabolites and process parameters were within accepted operating ranges, including glucose (2 g/L to 6 g/L after dropping from the initial value of approximately 8 g/L, FIG. 2B), lactate (less than approximately 2 g/L, FIG. 2C) and ammonia (less than approximately 5 mM, FIG. 2D).

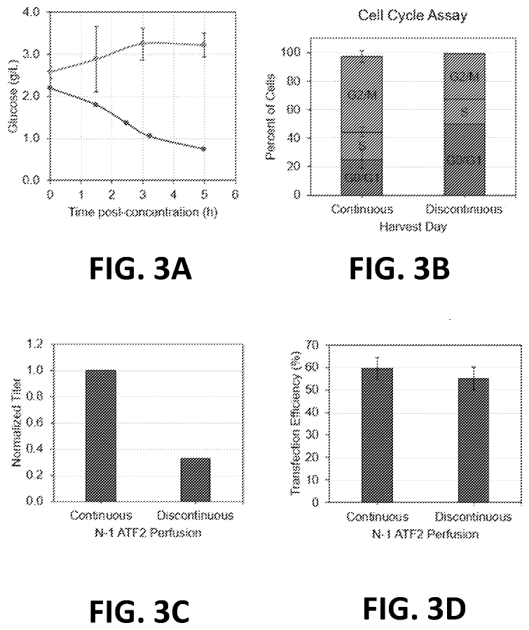

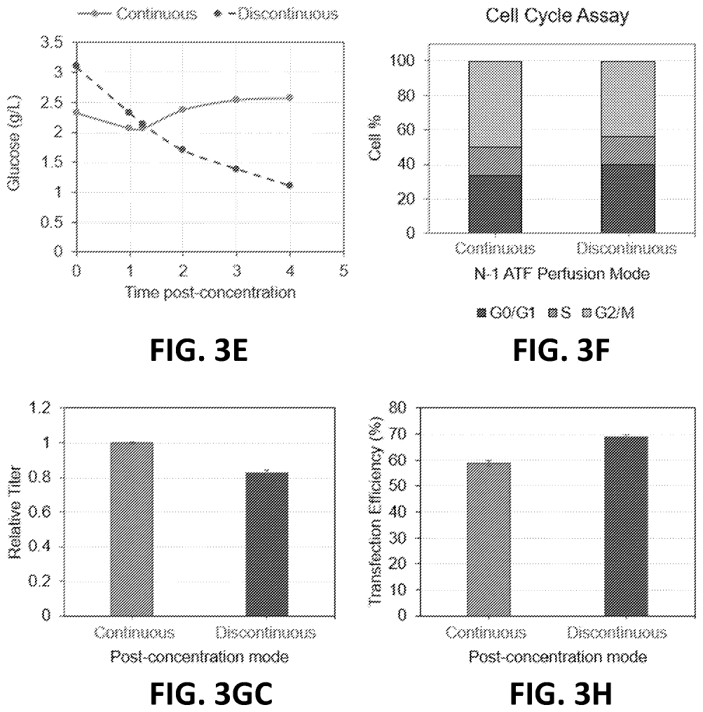

At day four, the cell density was increased to 90-100×10⁶ cells/mL using the ATF2 perfusion system to reduce working volumes and risk of contamination due to repeated open operations such as media exchange and centrifugation steps. Following cell density concentration, it was observed that glucose levels dramatically decreased due to higher nutrients demand of an intensified cell density culture (FIG. 3A). Because diminishing glucose levels in cell culture increase the risk of inducing quiescence, this scenario was undesirable for transfecting cells because actively proliferating cells take up recombinant DNA better than quiescent cells. It is thought that this phenomenon occurs because transport of DNA plasmids is more effective in replicating cells due to the disassembly of the nuclear envelope during mitosis causing the breakdown of the nuclear membrane barrier (Dean et al. (2005) Gene Ther. 12 (11): 881-890). Hence, to trigger high cell proliferation, perfusion process was implemented for cell cultures at high cell density (approximately 100×10⁶ cells/mL) and low working volumes to sustain nutrients within a safe cell culture range. Strategies to increase production titer via maintaining cells in logarithmic growth phase at the time of transfection has been successfully applied in several studies (Greene et al. (2021) Biotechnol. J. 16 (4): e2000251). However, a strategy focused on tuning the perfusion reactor post-concentration has not been reported. The all in-process attributes and performance for N−1 seed perfusion post-concentration are shown in FIGS. 3A-3D.

As seen in FIG. 3A, perfusing post-concentration at high cell density replenished glucose levels up to approximately 4 g/L preventing cells to transition to a quiescent state. In the perfused cell cultures, approximately 72% of the cells were in the replicating phases (S and G2/M) while the cell cultures without perfusion post-concentration (batch mode) only had 50% of cells in these phases (FIG. 3B). This difference in the percent of replicating cells had a substantial impact on titer in the production cultures. As seen in FIG. 3C, there is a three-fold reduction in titer in the batch mode as compared to the continuous mode.

To measure transfection efficiency, direct staining of surface IgG was conducted using a fluorescent anti-human IgG (H+L) antibody measured with a GUAVA® EASYCYTE™ flow cytometer. However, the transfection efficiencies between continuous and batch mode were comparable (FIG. 3D). This finding indicates that having a high population of replicating cells during transfection can contribute by other means besides more effective plasmid delivery to improve transient productivity. Another implication is that diminished glucose can condition the cell metabolism during the 14-day production run by inducing unfavorable transcriptional and metabolic changes.

To corroborate these data, the N−1 process with and without (discontinuous/batch) perfusion post-concentration was performed in parallel, and production was conducted post electroporation in shake flasks at higher seeding density (15e6/mL). N−1 perfusion (ATF2) was run for 4 days with a seeding density of 3-4e6/mL and a working volume of 3.5 L. Perfusion started on day 1 after cells reached approximately 8e6/mL. The perfusate rate was updated daily based on cell growth (VCD) and at a fixed CSPR of 0.04 nL/cell/day throughout the process. When cells reached approximately 60e6/mL on day 4, the perfusion reactor was concentrated to 90-100e6/mL for 40 min at a concentration rate of 24 ml/min. Following cell concentration, the perfusion was turned on (perfusion) or off (discontinuous/batch). FIGS. 3E-3F show the data for continuous and discontinuous/batch process. With the continuous system, the glucose levels were observed to be higher (FIG. 3E), and the percent of cells in the G2/M phase is higher compared to discontinuous/batch process (FIG. 3F).

FIGS. 3G and 3H show the performance of transient production in shake flasks using cells either from continuous or from discontinuous/batch process. From either process, the cells were inoculated at 15e6 vc/ml and followed the platform transient conditions (i.e., Temperature Shift on day 1 at 32° C., NaBu 1 mM, Basal media 1:1 B6:B9). The titer was 20% lower with the discontinuous/batch process indicating the perfusion of cells post concentration at N−1 stage maintains better cell health and hence improved cell culture performance post-electroporation (FIG. 3G.

Process Development Optimization in AMBR® 250 Bioreactor Lead to Titers Higher than 1 g/L

Following the findings of the shake flask screening, factors such as inoculation seeding density, addition of sodium butyrate (NaBu), temperature shift, and shift timing were evaluated in an AMBR® 250 bioreactor system. A custom Design of Experiments (DoE) design available in the JMP v15 software package with three blocks was carried out in a 24-run experiment.

TABLE 1
Effect summary report and significance for the AMBR ® 250 run

| Factor | FDR LogWorth | FDR P-value | Significance |
|---|---|---|---|
| Seeding Density | 5.02 | 1.00E−05 | ** |
| Temp. Shift Day | 3.4 | 4.00E−05 | ** |
| NaBu | 2.57 | 2.67E−03 | ** |
| Temp. Shift | 2.57 | 2.67E−03 | ** |
| Basal Media | 2.35 | 4.46E−03 | * |
| Block | 1.64 | 2.30E−02 | |
| P/V shift | 0.64 | 2.27E−01 | |

* α = 0.01;
** α = 0.001

To support the intensified fed-batch transient production, two media formulations, media A and media B, were evaluated. Media B was formulated for intensified cell culture manufacturing, and it differed from media A in the concentration of multiple individual components, including addition or removal of some components and increasing or decreasing concentration for others.

In addition to the six process parameters, in the AMBR® 250 bioreactors, agitation rates post-transfection was also evaluated since higher agitation rates can influence hydrodynamic stress (Sieack et al. (2013) J. Biotechnol. 161 (1): 41-49). To define the optimal operating range, a shift in power input/volume ratio (P/V), also known as averaged volumetric power input or power density, at 24 hours post-transfections was evaluated to minimize stress due to electroporation-induced cell sensitization (Pakhomova et al. (2011) PLoS One 6 (2): e17100). Here, a P/V shift was imposed to increase the agitation speed from 350 rpm to 450 rpm. Since three MAXCYTE™ CL2® electroporation processing assemblies (PAs) are required to inoculate all 24 AMBR® 250 bioreactors with transfected cells, a block effect was assigned to the batch of electroporated cells. The levels of each of the six factors, including the block effect, are shown in TABLE 1 and the final titer was used to identify optimum conditions.

Statistical analysis indicated that five of the six parameters evaluated were statistically significant (p<0.01) with only the P/V shift not having a significant effect on titer (TABLE 1). The block effect corresponding to the electroporation batch was found not significant (p=0.023). Among the four significant process parameters, the seeding density exhibited the greatest impact on titer as seen by the magnitude of the estimate in the statistical test.

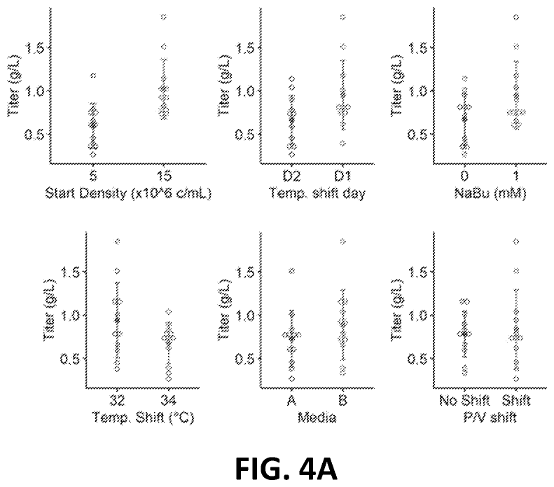

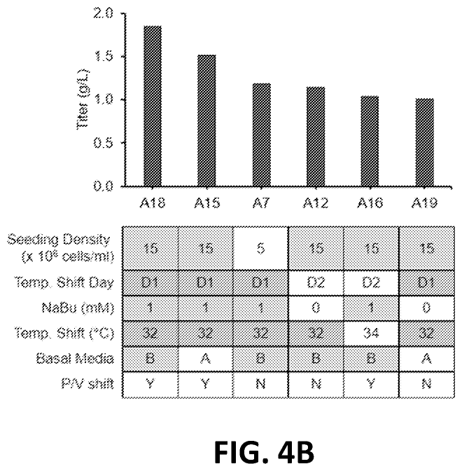

Six reactor conditions achieved titers greater than 1 g/L in a 14-day process (A18, A15, A7, A12, A16, and A19), and the maximum titer achieved was 1.85 g/L (A18) (FIG. 4B). Moreover, at least two conditions (A18 and A15) achieved titers greater than 1 g/L in only 7 days

FIG. 4A shows the main effects between level means for each of the six process parameters. Consistent with the shake flask screening, the seeding density of 15×10⁶ cells/mL had a higher titer than 5×10⁶ cells/ml. Temperature shift at day 1 was more beneficial for higher titer than shift at day 2, and 32° C. shift favors increased monoclonal antibody (mAb) expression than 34° C. Moreover, addition of 1 mM NaBu promoted higher mAb production than cell cultures without NaBu supplementation. Lastly, the media for intensified cell cultures (media B) facilitated higher titers of mAb than media A. Overall, these results indicated that higher seeding density, day 1 temperature shifts at 32° C., addition of 1 mM NaBu and the media formulation B enhanced transient expression of mAbs. Interestingly, the reactor with this unique combination of optimal levels, reactor A18, had the maximum titer at both day 7 and 14 (1.1 g/L and 1.85 g/L respectively). This result indicated that the selection of these conditions provided an adequate environment for high transient expression of mAbs.

Moreover, it was observed that the effects closely follow a linear behavior. The substitution of one optimal level for the suboptimal level, for example, by replacing the basal media B for media A had a detrimental effect on titer, reducing it 18% from 1.85 to 1.51 g/L (FIG. 4B). Similarly, reducing the seeding density from 15×10⁶ to 5×10⁶ cells/mL resulted in a 36% lower titer (1.85 vs 1.18 g/L).

During transient gene expression, temperature lower than 37° C. was been implemented to shift the cell state from proliferative to protein production and thus resulted in increased final titer. In addition, the reduced culture temperature benefited specific nutrient consumption and reduced accumulation of secondary metabolites. The timing of the shift can considerably affect transgene expression. The time point when the temperature shift was introduced was cell- and process-dependent, for this reason, it was desired to fully optimize the temperature shift strategy. The current data indicated that introducing temperature shift on day 1 was advantageous. This strategy worked well for intensified cell culture processes since peak VCD was reached earlier and thus it enabled more extended protein production phases.

Addition of NaBu had a multi-functional effect on transient gene expression including inhibition of cell proliferation and induction of gene accessibility and expression. Inhibition of cell cycle appeared to be a consequence of gene regulation in response to NaBu. After NaBu treatment, several genes associated with cell cycle control are known to be differentially expressed in CHO cells, including upregulation of three anti-proliferative genes, B cell translocation gene 3 (btg3), tumor susceptibility gene 101 (Tsg101) and cyclin dependent kinase 3 (Cdkn3). Moreover, other key cell cycle regulators such as c-myc and p21 are known to be down-regulated and up-regulated respectively after NaBu addition in human cell lines. Additionally, as a histone deacetylase inhibitor, NaBu is known to induce hyperacetylated histones that facilitate relaxed chromatin complexes, resulting in more accessible chromatin to transcription factors and hence, improving gene expression.

### Successful Scale-Up of the Transfection Process in 5-L Reactors

After demonstrating successful transient expression of the model mAb up to 1.85 g/L in the AMBR® 250 bioreactor system, the optimal condition (reactor A18) was scaled up in Finesse 5-L bioreactors (TABLE 2). Impeller tip velocity was used to scale up the agitation to match the cell growth and protein productivity (TABLE 2)

TABLE 2
Comparison of the transient process platform at AMBR ® 250
and 5-L bioreactor scales

| Process Parameter | AMBR ® 250 (Reactor A18) | 5-L bioreactor |
|---|---|---|
| Production media | Media B | Media B |
| Agitation speed | 350 rpm and then 450 rpm at D 1 | 150 rpm and then |
| | | 260 rpm at D 1 |
| Tip speed | 0.48 m/s −> 0.61 m/s | 0.5 −> 0.67 m/s |
| Temperature | 36.5° C. and then 32° C. at D 1 | |
| pH dead band | 7.1 ± 0.3 | |
| Glucose | To 4 g/L when <3 g/L | |
| Starting seeding | 15 × 10⁶ cell/mL | |
| density | | |
| Feed strategy | Every day from D 1-D 7, | |
| | then every other day | |
| NaBu | 1 mM | |
| DMA | 0.125% (v/v) | |

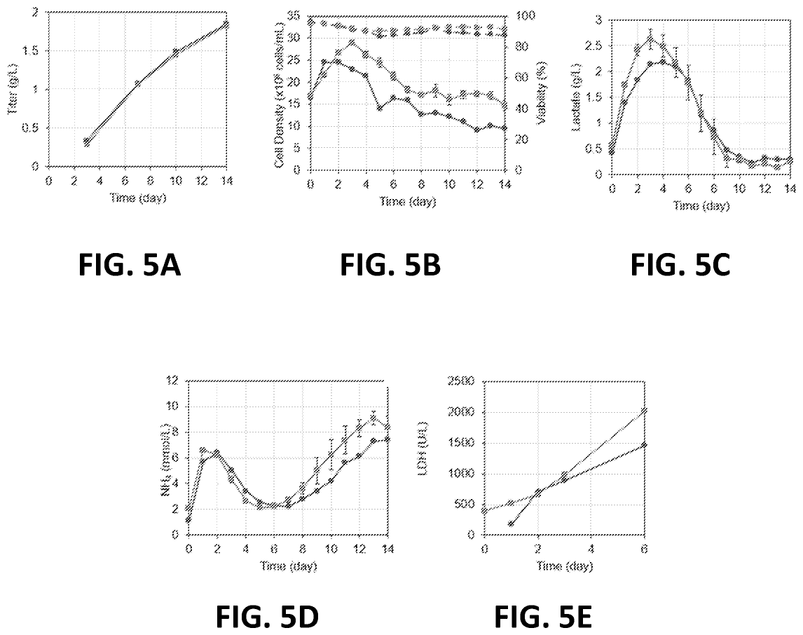

The tip velocity model for scale-up is often used in situations where limiting cell damage via shear stress to the cells is more important than changes in the mixing and oxygen transfer regimen. The fed-batch production cultures were monitored daily to determine viable cell count, viability, and metabolite levels (FIGS. 5A-5E). Overall, the cell culture performance was comparable in the two scales, indicating successful scale-up from AMBR® 250 to 5-L bioreactors.

Transfection efficiency was determined based on surface IgG staining at day 2 post-transfection with fluorescent anti-human IgG (H+L) measured with a GUAVA® EASYCYTE™ flow cytometer. Similar transfection efficiencies were achieved at both scales. Moreover, mAb expression was comparable between scales achieving a maximum titer of 1.85 g/L in a 14-day process. VCD peaked higher and later in the 5-L reactor while the viability in both scales remained above 90% throughout the 14 days. The higher VCD indicated a better hydrodynamic environment in the 5-L reactor as compared to the AMBR® 250.

The lactate and ammonia profiles followed the same trends even though the lactate was higher in the 5-L reactor, which may be attributed to the higher VCD in the latter scale. Overall, the metabolic profiles showed an ideal behavior, peaking during the growth phase, followed by a consumption of lactate and ammonium, a behavior typically observed in high productivity stable cell cultures.

Released LDH levels were also measured as a marker of cell damage for 6 days post-transfection. Low LDH levels were found between 24 and 48 hours post-transfection, indicating that the transfection conditions were gentle and hence successful implementation of the agitation strategy during the scale-up the process. LDH levels were slightly higher in the 5-L scale runs, which was attributed to the higher VCDs achieved in the 5 L bioreactors. When the LDH levels were normalized by total cell density, the LDH trends were very proportionally comparable at both scales.

Apoptosis analysis indicated that the decrease of VCD and increase of LDH release rates over the production period was due to programed cell death. As seen in FIGS. 5A-5E, the population of early and late apoptotic cells increased over time. However, at least 50% of the population remained healthy during the peak of production up to day 10.

A transition in cell cycle from largely dividing population (G2/S and M determined at transfection was approximately 80%) to largely residing in the non-dividing G0/G1 phase (over 50%) was observed by day 3. This shift to protein production phase coincided with the implementation of temperature shift and addition of NaBu 24 h post-transfection. Importantly, the G0/G1 population associated with cells at the production phase remained relatively constant throughout the remaining days of cell culture.

### Transient Material was Representative of Stable Clonal Material

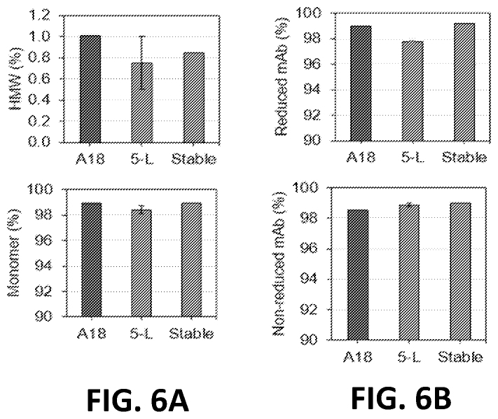

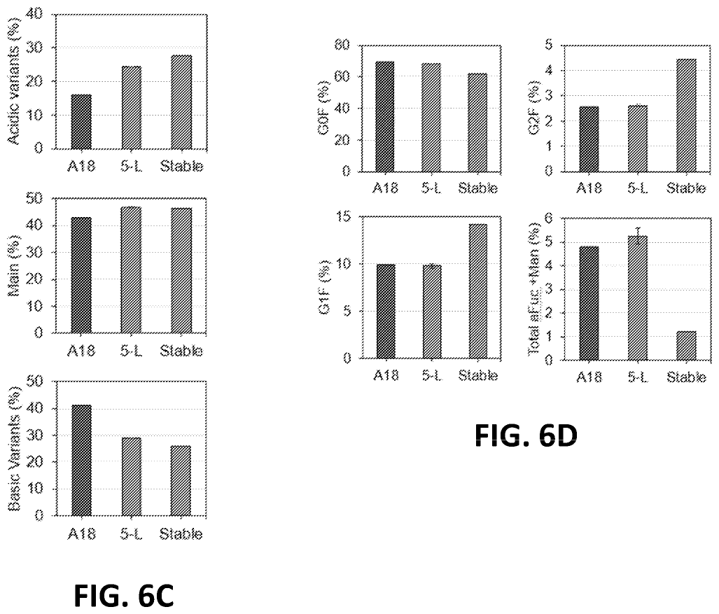

FIGS. 6A-6D display the general product quality attributes (PQA) of the purified mAb material. FIG. 6A and FIG. 6B displayed general purity as a function of size-exclusion chromatography and Caliper CE-SDS capillary electrophoresis. Material generated at 250 mL (AMBR® 250-A18) and 5 L scale was compared to the clonal material. Specifications displayed were based on general acceptance criteria for the model mAb used in this work. Low levels of HMW and LMW impurities were present, with higher than 98% of the material existing as intact monomer. Additionally, CE-SDS data showed that the material was intact under reducing conditions, with HC:LC ratio of 2:1 as expected. Charge variant profiles (FIG. 6C) showed the greatest variance in both acidic and basic species, with 10-20% deviation from clonal material profile. Overall, the transient material produced met release specifications. FIG. 6D outlines the fucosylated glycoforms and the total afucosylated species. Transient material was found to be comparable with the exception of afucosylated material, which was lower in the clonal material.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described herein.

All publications, patent applications, patents, and other references mentioned herein are incorporated by reference in their entirety. Database entries and electronic publications disclosed in the present disclosure are incorporated by reference in their entireties. The version of the database entry or electronic publication incorporated by reference in the present application is the most recent version of the database entry or electronic publication that was publicly available at the time the present application was filed. The database entries corresponding to gene or protein identifiers (e.g., genes or proteins identified by an accession number or database identifier of a public database such as Genbank, Refseq, or Uniprot) disclosed in the present application are incorporated by reference in their entireties. The gene or protein-related incorporated information is not limited to the sequence data contained in the database entry. The information incorporated by reference includes the entire contents of the database entry in the most recent version of the database that was publicly available at the time the present application was filed. In case of conflict, the present specification, including definitions, will control. In addition, the materials, methods, and examples are illustrative only and not intended to be limiting.

What is claimed is:

1. A method for high-yield and/or high quality production of a recombinant protein in a large scale bioreactor comprising
(i) culturing eukaryotic cells in a medium to concentrate the eukaryotic cells to a high density;
(ii) transiently transfecting the eukaryotic cells with a polynucleotide encoding the recombinant protein using electroporation.

2. The method of claim 1, wherein the eukaryotic cells are mammalian cells.

3. The method of claim 2, wherein the mammalian cells are selected from the group consisting of CHO, VERO, BHK, HEK, HeLa, COS, MDCK, and hybridoma cells.

4. The method of claim 3, wherein the mammalian cells are CHO cells.

5. The method of any one of claims 1 to 4, wherein the recombinant protein comprises an antibody or an antigen-binding portion thereof.

6. The method of claim 5, wherein the antibody or antigen-binding fragment binds an antigen selected from the group consisting of PD-1, PD-L1, CVTLA-4, LAG-3, TIGIT, GITR, CXCR4, CD73, HER2, VEGF, CD20, CD40, CD11a, tissue factor (TF), PSCA, IL-8, EGFR, HER3, and HER4.

7. The method of any one of claims 1 to 6, wherein the bioreactor is a high-throughput bioreactor.

8. The method of claim 7, wherein the high-throughput bioreactor is an AMBR®250 or an AMBR®15 bioreactor.

9. The method of any one of claims 1 to 8, wherein the bioreactor is a 1 L, 2 L, 5 L, 10 L, 25 L, 50 L, 100 L, 500 L, 1000 L, 2000 L, 5000 L, 10,000 L, or 20,000 L bioreactor.

10. The method of any one of claims 1 to 9, wherein the bioreactor is a fed-batch production bioreactor.

11. The method of any one of claims 1 to 9, wherein the culturing is perfusion culture.

12. The method of claim 11, wherein the perfusion is conducted by tangential flow filtration.

13. The method of claim 12, wherein the tangential flow filtration is alternating tangential flow filtration (ATF).

14. The method of claim 13, wherein the perfusion is conducted using a REPLIGEN™ ATF2 system.

15. The method of any one of claims 1 to 14, wherein the cells are concentrated to a density of at least about 40×10⁶, at least about 50×10⁶, at least about 60×10⁶, at least about 70×10⁶, at least about 80×10⁶, at least about 90×10⁶, at least about 100×10⁶, at least about 110×10⁶, at least about 120×10⁶, at least about 130×10⁶, at least about 140×10⁶, at least about 150×10⁶, at least about 160×10⁶, at least about 170×10⁶, at least about 180×10⁶, at least about 190×10⁶, or at least about 200×10⁶ cells/mL.

16. The method of any one of claims 1 to 14, wherein the cells are concentrated to a density of about 40×10⁶ to about 200×10⁶, about 60×10⁶ to about 200×10⁶, about 80×10⁶ to about 200×10⁶, about 100×10⁶ to about 200×10⁶, about 120×10⁶ to about 200×10⁶, about 140×10⁶ to about 200×10⁶, about 160×10⁶ to about 200×10⁶, about 180×10⁶ to about 200×10⁶, about 40×10⁶ to about 180×10⁶, about 60×10⁶ to about 180×10⁶, about 80×10⁶ to about 180×10⁶, about 100×10⁶ to about 180×10⁶, about 120×10⁶ to about 180×10⁶, about 140×10⁶ to about 180×10⁶, about 160×10⁶ to about 180×10⁶, about 40×10⁶ to about 160×10⁶, about 60×10⁶ to about 160×10⁶, about 80×10⁶ to about 160×10⁶, about 100×10⁶ to about 160×10⁶, about 120×10⁶ to about 160×10⁶, about 140×10⁶ to about 160×10⁶, about 40×10⁶ to about 140×10⁶, about 60×10⁶ to about 140×10⁶, about 80×10⁶ to about 140×10⁶, about 100×10⁶ to about 140×10⁶, about 120×10⁶ to about 140×10⁶, about 40×10⁶ to about 120×10⁶, about 60×10⁶ to about 120×10⁶, about 80×10⁶ to about 120×10⁶, about 100×10⁶ to about 120×10⁶, about 40×10⁶ to about 100×10⁶, about 60×10⁶ to about 100×10⁶, about 80×10⁶ to about 100×10⁶ cells/mL.

17. The method of any one of claims 1 to 14, wherein the cells are concentrated to a density of about 100×10⁶, about 110×10⁶, about 120×10⁶, about 130×10⁶, about 140×10⁶, about 150×10⁶, about 160×10⁶, about 170×10⁶, about 180×10⁶, about 190×10⁶, about 200×10⁶, about 210×10⁶, about 220×10⁶, about 230×10⁶, about 240×10⁶, or about 250×10⁶ cells/mL.

18. The method of any one of claims 1 to 14, wherein electroporation is conducted at a cell density of about 100×10⁶, about 110×10⁶, about 120×10⁶, about 130×10⁶, about 140×10⁶, about 150×10⁶, about 160×10⁶, about 170×10⁶, about 180×10⁶, about 190×10⁶, about 200×10⁶, about 210×10⁶, about 220×10⁶, about 230×10⁶, about 240×10⁶ or about 250×10⁶ cells/mL.

19. The method of any one of claims 1 to 18, wherein the electroporation is flow-based electroporation.

20. The method of any one of claims 1 to 18, wherein electroporation is conducted using a MAXCYTE™ transfection system.

21. The method of claim 20, wherein the transfection system is a MAXCYTE™ STX transfection system.

22. The method of any one of claims 1 to 21, wherein electroporation is conducted at a DNA to cell ratio of about 1, about 1.1, about 1.2, about 1.3, about 1.4, about 1.5, about 1.6, about 1.7, about 1.8, about 1.9, or about 2 μg DNA per 1×10⁶ cells.

23. The method of any one of claims 1 to 22, wherein the post-transfection cell culture start density is between 5×10⁶ and 20×10⁶ cells/mL.

24. The method of any one of claims 1 to 23, wherein the temperature of the post-transfection cell culture is shifted for some of the culture period.

25. The method of claim 24, wherein the post-transfection cell culture comprises a temperature shift on Day 1 or Day 2.

26. The method of claim 25, wherein the temperature shift is from about 37° C. to about 32° C.

27. The method of claim 25, wherein the temperature shift is from about 37° C. to about 34° C.

28. The method of any one of claims 1 to 27, wherein the post-transfection cell culture comprises the addition of N,N-dimethyl acetamide (DMA).

29. The method of claim 28, wherein DMA is added at about 0.125% v/v or about at 0.250% v/v.

30. The method of claim 28, wherein DMA is added at between about 0.125% v/v to about 0.250% v/v.

31. The method of any one of claims 1 to 30, wherein the post-transfection cell culture comprises the addition of sodium butyrate (NaBu.)

32. The method of claim 31, wherein NaBu is added at a concentration between about 1 mM and about 2 mM.

33. The method of any one of claims 1 to 32, wherein protein yield of the method is at least about 0.1 g/L, at least about 0.2 g/L, at least about 0.3 g/L, at least about 0.4 g/L, at least about 0.5 g/L, at least about 0.6 g/L, at least about 0.7 g/L, at least about 0.8 g/L, at least about 0.9 g/L, at least about 1 g/L, at least about 2 g/L, at least about 3 g/L, at least about 4 g/L, at least about 5 g/L, or at least about 6 g/L after 7 days of culture.

34. The method of any one of claims 1 to 33, wherein the protein yield is up to 2 g/L after 14 days of culture.

35. A method for high-yield and/or high quality production of a recombinant protein in a bioreactor comprising
(v) culturing eukaryotic cells under continuous ATF perfusion prior to transfection,
(vi) concentrating the cells to a density of 40-200×10⁶ cells/mL using continuous ATF perfusion prior to transfection;
(vii) transiently transfecting the eukaryotic cells with a polynucleotide encoding the recombinant protein using flow-based electroporation; and.
(viii) post-transfection culturing the transiently transfected eukaryotic cells.

36. The method of claim 1 or claim 35, wherein post-transfection culturing comprises (i) a cell culture start density of about 15×10⁶ cells/mL, (ii) DMA at about 0.125% (v/v/), (iii) NaBu at about 1 mM, and (iv) a temperature shift at Day 1 from about 36.5° C. to about 32° C., wherein the volume of the bioreactor is between about 250 mL and about 5 L.

37. The method of any one of claims 1 to 36, wherein the values of product quality attributes of the recombinant protein obtained by transiently transfecting the eukaryotic cells is with +/−10% of the values of product quality attributes of the recombinant protein obtained by stable transfection.

38. The method of claim 37, wherein the quality attributes are selected from the group consisting of
(i) protein aggregation;
(ii) reduced and non-reduced species;
(iii) charge variants;
(iv) glycosylation profile; and,
(v) any combination thereof.

39. The method of claim 38, wherein the protein aggregation quality attributes are selected from the group consisting of (i) percentage of high molecular weight species (HMW %), (ii) percentage of monomeric species, and (iii) any combination thereof.

40. The method of claim 39, wherein the protein aggregation quality attributes are determined using HPLC size exclusion chromatography.

41. The method of claim 38, wherein the reduced and non-reduced species quality attributes are selected from the group consisting of (i) percentage of reduced recombinant protein, (ii) percentage of non-reduced recombinant protein, and (iii) any combination thereof.

42. The method of claim 41, wherein the reduced and non-reduced species quality attributes are determined using capillary electrophoresis under reducing and non-reducing conditions (CE-SDS).

43. The method of claim 38, wherein the charge variants quality attributes are selected from the group consisting of (i) percentage of basic variants, (ii) percentage of acidic variant, (iii) percentage of main species, and (iv) any combination thereof.

44. The method of claim 43, wherein the charge variants quality attributes are determined by analyzing isoelectric distribution by capillary isoelectric focusing (iCIEF).

45. The method of claim 38, wherein the glycosylation profile comprises one or more N-linked glycans.

46. The method of claim 42, wherein the N-linked glycans comprise (mannose-3-N-acetylglucosamine-4-fucose) (G0F), mannose-3-N-acetylglucosamine-4-galactose-1-fucose (G1F), mannose-3-N-acetylglucosamine-4-galactose-2-fucose (G2F), mono-sialylated mannose-3-N-acetylglucosamine-4-galactose-1-fucose (S1G1F), mono-sialylated mannose-3-N-acetylglucosamine-4-galactose-2-fucose (S1G2F), mono-sialylated mannose-3-N-acetylglucosamine-4-galactose-3-fucose (S2G3F), di-sialylated mannose-3-N-acetylglucosamine-4-galactose-2-fucose (S2G2F), or any combination thereof.

47. The method of claim 38, wherein the glycosylation profile quality attributes are selected from the group consisting (i) percentage of G1F, (ii) percentage of G0F, (iii) percentage of G2F, (iv) percentage of total afucosylated protein, and (v) any combination thereof.

48. The method claims 45 to 47, wherein the glycosylation profile quality attributes are determined using HPLC methods.

49. The method of claim 48, wherein the HPLC method is Ultra Performance Liquid Chromatography with fluorescence detection (UPLC-FLR).

50. The method of any one of claims 1 to 48, wherein the medium in (i) comprises glucose at a concentration sufficient to maintain a high cell density.

51. The method of claim 50, wherein the concentration of glucose is at least about 0.1 g/L, at least about 0.5 g/L, at least about 1.0 g/L, at least about 1.5 g/L, at least about 2.0 g/L, at least about 2.5 g/L, at least about 3.0 g/L, at least about 3.5 g/L, at least about 4.0 g/L, at least about 4.5 g/L, or at least about 5.0 g/L.

52. A recombinant protein obtained according to the method of any one of claims 1 to 51.

53. The recombinant protein of claim 52, wherein the recombinant protein is antibody or antigen-binding fragment binds an antigen selected from the group consisting of PD-1, PD-L1, CVTLA-4, LAG-3, TIGIT, GITR, CXCR4, CD73, HER2, VEGF, CD20, CD40, CD11a, tissue factor (TF), PSCA, IL-8, EGFR, HER3, and HER4.

54. A pharmaceutically composition comprising a recombinant protein obtained according to the method of any one of claims 1 to 51.

55. A cell or plurality thereof obtained according to the method of any one of claims 1 to 51.

56. The cell or plurality of cells of claim 55, wherein the cells is Chinese hamster ovary (CHO) cell.

57. A bioreactor for the manufacture of a recombinant protein produced according to the method of any one of claims 1 to 51.

58. A bioreactor comprising the cell or plurality of cells of claim 56 or 57.

59. A system for high-yield production of a recombinant protein by transient transfection comprising:
(i) a bioreactor;
(ii) an ATF perfusion system; and,
(iii) an electroporation transfection system.

60. A method to accelerate or shorten the development timeline of a recombinant protein comprising
(i) culturing eukaryotic cells in a medium to concentrate the eukaryotic cells to a high density, and
(ii) transiently transfecting the eukaryotic cells with a polynucleotide encoding the recombinant protein using electroporation.

61. A method to reduce the host cell toxicity of a recombinant protein comprising
(i) culturing eukaryotic cells in a medium to concentrate the eukaryotic cells to a high density, and
(ii) transiently transfecting the eukaryotic cells with a polynucleotide encoding the recombinant protein using electroporation.

62. A method to produce a recombinant protein for individualized therapeutics comprising
(i) culturing eukaryotic cells in a medium to concentrate the eukaryotic cells to a high density, and
(ii) transiently transfecting the eukaryotic cells with a polynucleotide encoding the recombinant protein using electroporation.
